(19) United States
(12) Patent Application Publication
(10) Pub. No.: US 2008/0234184 A1
(43) Pub. Date: Sep. 25, 2008

(54) Vectors For the Co-Expression of Membrane Domains of Viral Envelope Proteins and Uses Thereof

(75) Inventors: Pierre Falson, Sainte Foy les Lyon (FR); Cedric Montigny, Forges-les-Bains (FR); Francois Penin, Decines (FR)

Correspondence Address: MCKENNA LONG & ALDRIDGE LLP, 1900 K STREET, NW, WASHINGTON, DC, 20006, US

(73) Assignees: CENTRE NATIONAL DE LA RECHERCHE SCIENTIFIQUE, 75794 PARIS CEDEX (FR); COMMISSARIAT A L'ENERGIE ATOMIQUE, 75752 PARIS 15 EME (FR)

(21) Appl. No.: 10/569,882

(22) PCT Filed: Aug. 19, 2004
(86) PCT No.: PCT/FR04/50385

§ 371 Date: Jun. 16, 2006

(30) Foreign Application Priority Data
Aug. 28, 2003 (FR) 0350470

(51) Int. Cl. A61K 38/00; C12N 15/00; C12N 1/20; A61P 43/00; C12P 21/04; C07K 14/00

(52) U.S. Cl. 514/12; 435/320.1; 435/252.3; 435/252.33; 435/69.1; 530/324

(57) ABSTRACT

The present invention discloses a vector for the coexpression of membrane domains of the envelope proteins of a virus, and also a method for producing homo- and/or hetero-oligomers of these domains. This vector comprises at least one region for replication and for maintenance of said vector in the host cell; a first region consisting successively, in said direction of translation of the vector, of a first promoter followed by a first sequence encoding a first chimeric protein comprising in particular a sequence encoding one of said at least two membrane domains; and a second region consisting successively, in said direction of translation of the vector, of a second promoter followed by a second sequence encoding a second chimeric protein comprising in particular a sequence encoding the other of said at least two membrane domains. The present invention is useful for the production of medicinal products for the treatment or prophylaxis of hepatitis C.

### BACKGROUND OF THE INVENTION

1. Field of the Invention

The present invention relates to a vector for the coexpression of membrane (transmembrane) domains of envelope proteins of a virus, and also to a method for producing homo- and/or hetero-oligomers of these domains. These membrane domains are domains of viral envelope proteins that allow viruses to anchor to the target cells that they will infect. The vector allows coexpression of the TME1 and TME2 membrane domains of the hepatitis C virus envelope proteins, and the production of homo- and/or hetero-oligomers of these domains.

In the description that follows, reference numbers appear between square brackets [ ] and refer to the numbers in the attached “List of References.”

2. Description of the Background Art

The determination of the three-dimensional (3D) structure is a decisive step in understanding the structure and function of proteins. For this, it is necessary to be able to produce sufficient amounts of the proteins for study, preferably in their (native) functional conformation. Great efforts and means have been, and are being, expended to achieve this aim, and these efforts have increased with the accumulation of data provided by genome sequencing programs.

In this context, bacteria are a widely used by the scientific community as a means of production. The overexpression of proteins in bacteria is not, however, without problems. Specifically, it most commonly gives rise to the following two scenarios.

The first and most common case, is that in which the protein is overproduced and in aggregated form as inclusion bodies. This concerns polytypic proteins and/or large proteins. In this case, the kinetics of folding of the protein is clearly slower than its rate of biosynthesis. This promotes exposure of the hydrophobic regions of the protein that are normally buried to the aqueous solvent, generating non-specific interactions that result in the formation of insoluble aggregates. Depending on the degree of disorder of this folding, the inclusion bodies can be solubilized/unfolded under non-native conditions, by using urea or guanidine. The solubilized protein is subsequently subjected to various treatments, such as dialysis or dilution, to obtain, in some cases only, proteins in their native 3D folding.

The second case is that in which the expression engenders varying degrees of toxicity, ranging from an absence of expression product if the host cell manages to adapt, to the death of the cell if the product is too toxic. This occurs quite frequently, and most commonly with proteins or membrane domains or domains of membrane proteins, such as, for example, envelope proteins of the hepatitis C virus (HCV) [1] or of the human immunodeficiency virus (HIV) [2].

The problem of host cell toxicity for concerns essentially the expression of membrane proteins, i.e., proteins having a hydrophobic domain, which are of growing interest. They are, first, relatively numerous based on the sequencing of various genomes confirming that they represent approximately 30% of the proteins potentially encoded by these genomes [3]. Second, they constitute 70% of the therapeutic targets and their alteration is a cause of numerous genetic diseases [4].

It is therefore essential to develop methods that facilitate or allow the expression of such proteins or of their membrane portions.

Efforts in this direction include, for example, the development of bacterial strains that either are more tolerant of the expression of membrane proteins [5,6], or more strictly regulate the mechanism expression, as in the case of the E. coli strain BL21 (DE3)pLysS developed by Stratagene. However, these improvements still do not eliminate the phenomenon of toxicity in all cases, in particular when hydrophobic peptides corresponding to membrane anchors are expressed.

One of the major medical conditions in which the stakes are currently highest is hepatitis C which is caused by the HCV of the family flaviviridae which specifically infects hepatic cells [7]. HCV infects 170 million humans throughout the world, and it is estimated that 75% of seropositive individuals develop chronic infections [8]. This virus consists of a positive strand RNA of approximately 9500 bases that encodes a 3033 residue polyprotein [9], represented in FIG. 1. After expression, the polyprotein is cleaved by endogenous and form the viral envelope.

During the virus maturation process, the E1 and E2 proteins associate to form hetero-oligomers, which have not yet been fully characterized. E1 and E2 each consist of an ectodomain (“ed” in FIG. 1) and a C-terminal region, rich in hydrophobic amino acids, which forms a transmembrane domain (“TM” in FIG. 1; referred to herein usually as “membrane domain”) that anchors the proteins to the endoplasmic reticulum membrane [10]. Each of the ectodomains and also the membrane domains [11] are involved in the phenomenon of oligomerization and influence the organization of the virus envelope. Because they are involved in the process of oligomerization of the E1 and E2 proteins, the TME1 and TME2 membrane domains are highly advantageous potential therapeutic targets.

Various attempts to express the E1 or E2 proteins in E. coli [12, 13] or in sf9 insect cells infected with baculovirus [14] have been unsuccessful because of the toxicity resulting from their expression. This toxicity is essentially generated by the membrane domains, and occurs quite frequently, most commonly with membrane proteins or domains of, for example, the envelope proteins of HCV [13] or HIV [15].

To date, the existing recombinant expression systems do not enable production of these membrane proteins. Furthermore, when transmembrane domains, for example HCV TME1 or TME2, are obtained, and they appear as a mixture, but never reproduce the native association states of the proteins as they occur in the viral envelope.

There exists, therefore, a real need for a system for producing membrane domains that cooperate in their native, functional conformation in the viral envelope, in particular as they generate the envelope and/or mediate viral recognition and/or binding to its target cell. It is also desirable that this system allow the domains produced to mimic their various association states during (a) the genesis of the virus envelope and/or (b) as the envelope functions in the processes of viral target cell recognition and/or binding.

Such a system would, for example, enable large scale testing of chemical and biological compounds, for example peptides, for their ability to disturb the formation of the various association states of the membrane domains, which could therefore interfere with formation of the virus and/or its action in recognizing its target cells.

### SUMMARY OF THE INVENTION

The present invention relates to a vector for the coexpression of membrane domains of envelope proteins of a virus, and also to a method for producing homo- and/or hetero-oligomers of these domains. These membrane domains are domains of viral envelope proteins that allow viruses to anchor to the target cells that they will infect.

The vector of the present invention allows, for example, the coexpression of the TME1 and TME2 membrane domains of the HCV envelope proteins, and the production of homo- and/or hetero-oligomers of these TME1 and TME2 domains.

The present invention provides a vector that enables, in general, recreation of various association states of the membrane domains of viral envelope proteins during the constitution thereof.

This vector allows large scale testing of chemical or biological compounds, for example peptides, capable of disturbing the formation of the various association states of the membrane domains of viral envelope proteins, and therefore potentially of disturbing viral formation or binding of the virus to its target host cells.

The present invention therefore also provides a screening method for identifying chemical or biological compounds that interfere with formation of the various association states of the membrane domains of viral envelope proteins. It therefore finds many applications, particularly for research of mechanisms of viral infection and in the search for, and development of, novel active compounds to combat viral infections.

### ABBREVIATIONS

E. coli: Escherichia coli. DP: aspartate-proline (Asp-Pro) dipeptide. GST: glutathione S-transferase. TrX: thioredoxin. HCV: hepatitis C virus. HIV: human immunodeficiency virus. TME1 and TME2: the two membrane or transmembrane domains of the HCV E1 and E2 envelope glycoproteins. PCR: polymerase chain reaction. LB (10 g tryptone, 5 g yeast extract, 5 g NaCl, q.s. 1 L H₂O). Amp: ampicillin. Kan: kanamycin. OD: optical density. LS: lysis solution (50 mM Tris-HCl, pH 8.0, 2.5 mM EDTA, 2% SDS, 4M urea, 0.7M β-mercaptoethanol). IPTG: isopropyl-1-thio-β-D-galactoside. aa: amino acid(s). PAGE: polyacrylamide gel electrophoresis. In various of the Figures: “Lmw”: low molecular weight markers; “G”: GST; “T”: TrX; “No induc.”: no induction; “Ab-TrX”: antibody specific for TrX; and “Ab-GST”: antibody specific for GST.

### BRIEF DESCRIPTION OF THE DRAWINGS

FIG. 1 is a diagrammatic representation of a portion of the HCV polyprotein and shows the amino acid sequences of the C-terminal transmembrane domains of the TME1 and TME2 envelope proteins. The letters at the top refer to the proteins constituting the polyprotein as follows: C— capsid protein. E1 and E2—HCV E1 and E2 envelope proteins; P7—HCV P7 protein. The E1 and E2 proteins are divided into the “ed” (ectodomain) and “TM” (transmembrane domain).

FIGS. 2A and 2C are photographs of two 12% polyacrylamide gels after PAGE demonstrating the separation by migration and Coomassie blue staining of, respectively, the GST-DP-TME1 and GST-DP-TME2 (FIG. 2A; GST is further abbreviated as “G”) and TrX-DP-TME1 and TrX-DP-TME2 (FIG. 2C; TrX is further abbreviated as “T”) chimeras obtained.

FIGS. 2B and 2D are photographs of PAGE gels of, respectively, FIGS. 2A and 2C, subjected to Western blotting (immunodetection) to reveal (1) GST chimeras with an antibody specific for GST demonstrating dimeric (2×) and trimeric (3×) forms of the GST-DP-TME1 and GST-DP-TME2 chimeras (FIG. 2) and, (2) the TrX chimeras with an antibody specific for TrX demonstrating monomeric (1×), dimeric 2×) and trimeric (3×) forms of the TrX-DP-TME1 and TrX-DP-TME2 chimeras (FIG. 2D).

FIG. 3 A is a photograph of a PAGE gel, demonstrating the GST-DP-TME2 and GST-DP-TME2-C731&C733A (also referred to in the text below as “C731/C733A”) chimeras and the oligomers thereof. FIG. 3B is a photograph of a PAGE gel demonstrating the TrX-DP-TME2 and TrX-DP-TME2-C731&C733A chimeras and the oligomers thereof. FIG. 3C is a photograph of a PAGE gel demonstrating the TrX-DP-TME1, TrX-DP-TME1_G354L, TrX-DP-TME1_G358L and TrX-DP-TME1_G354&358L (the double mutation also referred to in the text below as “G354/358L”) chimeras and the oligomers thereof.

FIG. 4 shows the introduction of the mutations and restriction sites for cloning pGEXKT to obtain a vector according to the invention and the oligonucleotides produced for amplifying the TME2-C731&733A fragment.

FIGS. 5A and 5B show coexpression of the GST and TrX chimeras fused, respectively, to the TME2 and TME1 membrane domains and effects of mutation of the cysteines in TME2 on their homo- and hetero-oligomerization. FIG. 5A shows a schematic diagram of a vector that coexpresses the GST-DP-TME2 and TrX-DP-TME1 chimera according to the present invention. FIG. 5B shows a Western blot demonstrating, by immunodetection with an anti-GST antibody, the expression of membrane proteins in E. coli BL21 Gold(DE3)pLysS bacteria transformed with one of the four vectors pGEXKT-DP-TME2_C731/C733A, pGEXKT-DP-TME2+TrX-DP-TME1, pGEXKT-DP-TME2_C731&C733A+TrX-DP-TME1 and pET32a-TrX-DP-TME1 of the present invention.

FIG. 6A-6B show coexpression of the GST and TrX chimeras fused, respectively, to the TME2 and TME1 membrane domains and the effect of mutation of the cysteines in TME2 on the homo- and hetero-oligomerization. FIG. 6A (see FIG. 5B) shows results of immunodetection carried out with an anti-TrX antibody. FIG. 6B is a diagrammatic representation of the organization of oligomers (also shown in FIG. 6A) using the chimeric proteins expressed from the vectors of the invention.

### DESCRIPTION OF THE PREFERRED EMBODIMENTS

The objective obtained by the present invention is precisely that of solving the abovementioned problems of the prior art and of satisfying the abovementioned needs by providing a system for the coexpression of membrane domains that cooperate or interact in their functional conformation in the envelope of a virus, in particular for the constitution of the virus envelope and/or for recognition and/or binding of the virus to its target cell.

The present invention is directed to a nucleic acid vector for the coexpression of at least two membrane domains of viral envelope proteins that cooperate in their native functional conformation in the virus envelope. The vector comprises:

at least one region for replication and for maintenance of said vector in the host cell;

a first region consisting sequentially or successively of, in the direction of translation of the vector, a first promoter followed by a first coding nucleotide sequence encoding a first chimeric protein, and which consists of, in the direction of translation of the vector, a first nucleotide sequence encoding a first soluble protein, a nucleotide sequence encoding an Asp-Pro dipeptide and a nucleotide sequence encoding one of the at least two membrane domains; and

a second region consisting sequentially of, in the direction of translation of the vector, a second promoter followed by a second nucleotide sequence encoding a second chimeric protein, the second sequence encoding the second chimeric protein consisting of, in the direction of translation of the vector, a second nucleotide sequence encoding a second soluble protein, a nucleotide sequence encoding an Asp-Pro dipeptide and a nucleotide sequence encoding the other of said at least two membrane domains.

The term “membrane protein domain” or “membrane domain” is intended to mean the portion of a viral envelope protein which is hydrophobic particularly in the part anchoring to the membrane of the target cells. It may of course be a whole protein, which is a membrane protein, or a membrane portion of a protein which also has a non-membrane hydrophilic domain.

According to the invention, advantageously, the first and the second regions are contiguous, but the arrangement of these two regions with respect to one another in the vector is, a priori, of no importance.

According to the invention, the first and second soluble proteins may be identical or different. They may be glutathione S-transferase (GST), thioredoxin (TrX) or any other equivalent soluble protein. The amino acid sequences of GST and of TrX are, for example, respectively the sequences SEQ ID NO:25 and SEQ ID NO:37. The nucleotide sequences encoding GST and TrX which can be used in the vector of the present invention to encode GST and TrX are, for example, respectively the sequences SEQ ID NO:24 and SEQ ID NO:36.

According to the invention, the nucleotide sequence encoding the Asp-Pro dipeptide may, for example, be gac-ccg or any other hexanucleotide sequence encoding this dipeptide.

The sequence encoding Asp-Pro (DP in single letter code), placed upstream of the nucleotide sequence encoding each membrane protein, makes it possible, entirely unexpectedly, to abolish the toxic effect of the co-expressed membrane proteins on the host cell. Furthermore, the inventors have noted that, entirely surprisingly, the elimination of toxicity of the protein in the host is even more effective when the membrane peptides are produced as a C-terminal fusion with a soluble protein, for example, CST or TrX, with the Asp-Pro coding sequence inserted between each soluble protein coding sequence and each membrane peptide coding sequence in the coexpression vector of the present invention.

The vector of the present invention allows overproduction, as coexpression, of at least two membrane domains of the viral envelope proteins that cooperate in their native functional conformation in the virus envelope. These are also membrane proteins in the host cells, and are, in particular, hydrophobic proteins, especially peptides which correspond to, or which comprise, hydrophobic domains of proteins which are capable of anchoring to host cell membranes. They may, for example, be membrane proteins or domains thereof. They may, for example, be viral envelope proteins, for example of HCV, of HIV or of any other virus that is pathogenic for humans or, in general, for mammals. In the present invention, these envelope proteins are reduced to their membrane domain, i.e., their hydrophobic domain. This is what the term “membrane domains” is intended to mean. The viruses with which the present invention is concerned are in fact all viruses which possess, in their structure, membrane proteins that interact in constituting the virus envelope and/or for recognition and/or binding of the virus to its target cell.

For example, in the case of HCV envelope proteins, the vector of the present invention may be a co-expression vector for the TME1 and TME2 membrane domains that allows the coexpression of the TME1 and TME2 domains, corresponding in particular to segments 347-383 and 717-746 of the polyprotein encoded by the virus RNA and having the following sequences:

TME1:

(SEQ ID NO:2

³⁴⁷MIAGAHWGVLAGIAYFSMVGNWAKVLVVLLLFAGVDA³⁸³
TME2:

(SEQ ID NO:16)

⁷¹⁷MEYVVLLFLLLADARVCSCLWMMLLISQAEA⁷⁴⁶

Thus, according to the invention, one of the two membrane domains may have a peptide sequence (amino acid sequence) selected from the group of sequences SEQ ID NO:2, SEQ ID NO:10, SEQ ID NO:12 and SEQ ID NO:14, and the other of the two domains has an amino acid sequence selected from group consisting of sequences SEQ ID NO:16 and SEQ ID NO:22.

The peptide sequences SEQ ID NO:10, SEQ ID NO:12 and SEQ ID NO:14 correspond to the amino acid sequence of TME1 (SEQ ID NO:2) comprising point mutations. The amino acid sequence SEQ ID NO:22 corresponds to the sequence of TME2 (SEQ ID NO:16) comprising two point mutations. The role of these point mutations in accordance with the present invention is explained below.

According to the invention, in the vector, the nucleotide sequence encoding one of said at least two membrane domains may, for example, be selected from the group of nucleotide sequences SEQ ID NO:1, SEQ ID NO:9, SEQ ID NO:11 and SEQ ID NO:13, and the nucleotide sequence encoding the other of said at least two membrane domains may be selected from the group of sequences SEQ ID NO:15 and SEQ ID NO:17. These nucleotide sequences encode, respectively, the amino acid sequences SEQ ID NO:2, SEQ ID NO:10, SEQ ID NO:12, SEQ ID NO:14, SEQ ID NO:16 and SEQ ID NO:22 mentioned above. Those skilled in the art will be able to readily determine other nucleotide sequences encoding such peptides or mutated peptides.

The TME1 and TME2 peptides are respectively produced as a C-terminal fusion of soluble proteins, for example GST and/or TrX, to form the chimeras, for example, GST-DP-TME1, GST-DP-TME2, TrX-DP-TME1 and TrX-DP-TME2.

For example, in the vector of the present invention, the first chimeric protein may be a protein having a sequence selected from the group of sequences SEQ ID NO:28, SEQ ID NO:43, SEQ ID NO:46, SEQ ID NO:49 and SEQ ID NO:52, and the second chimeric protein may be a protein having a sequence selected from the group of sequences SEQ ID NO:31, SEQ ID NO:34, SEQ ID NO:55 and SEQ ID NO:58. For the same reasons as those mentioned above, in particular for advantageously obtaining hetero-oligomers of the coexpressed proteins, when the first chimeric protein has the sequence SEQ ID NO:28, the other chimeric protein is different from SEQ ID NO:31, and vice versa.

The membrane proteins produced from the vector of the present invention form monomers, dimers, trimers and, to a lesser extent, multimeric forms, which are sometimes stable enough to withstand the denaturing conditions of separation on a polyacrylamide gel in the presence of the detergent sodium dodecyl sulfate (SDS).

The inventors have discovered, unexpectedly, for the TME1 and TME2 membrane proteins of the HCV envelope, that, whatever the forces of interaction that stabilized these oligomeric forms, they can be eliminated by either:
(1) the point mutations G354L and/or G358L in TME1 (the glycine at position 354 and/or 358 of TME1 is replaced with a leucine); and/or
the point mutations C731A and C733A (referred to interchangeably as “C731/C733A” or “C731&C733A”) in TME2 (the cysteines at position 731 and 733 of TME2 are replaced with an alanines).

According to the invention, it is not necessary to produce all the mutations of TME1 and of TME2 in the vector in order to eliminate the forces of interaction. The mutations of either TME1 or TME2 can be sufficient in the coexpression vector of the present invention to obtain this result. Thus, if specific hetero-oligomeric forms are desired, preferably, when one of the two domains is SEQ ID NO:2, the other domain is different from SEQ ID NO:16, and vice versa. Similarly, when one of the two domains is encoded by the sequences SEQ ID NO:1 (encoding TME1), the other coding domain is different from SEQ ID NO:15 (encoding TME2), and vice versa. Through the choice of the mutations, according to the invention, it is therefore possible to preferentially obtain certain hetero-oligomeric forms, or no homo- or hetero-oligomeric form.

For example, in a particular embodiment of the present invention, a vector was constructed by integrating a region encoding the chimera (soluble protein-DP-TME1) and a region encoding the chimera (soluble protein-DP-TME2). This coexpression allowed the formation, first, of the homo-oligomers observed with independent expressions (trials without coexpression) and, secondly, entirely surprisingly, of a heterodimer having the following arrangement:

{[soluble protein-DP-TME1]₁−[soluble protein-DP-TME2]₁} and

a heterotrimer having the following arrangement:
{-[soluble protein DP-TME1]₂-[soluble protein-DP-TME2]₁]}.
   Furthermore, using such a vector, but with the double mutation C731A/C733A in TME2, it is notable that the inventors were able to eliminate not only the hetero-oligomeric forms but also the [TrX-DP-TME1]₃ trimer.

Examples of TME1 mutated chimeric proteins according to the present invention are GST-DP-TME1_G354L (SEQ ID NO:65); GST-DP-TME1_G358L (SEQ ID NO:67); and GST-DP-TME1_G354/358L (SEQ ID NO:65), encoded, for example, respectively by the oligonucleotides of sequences SEQ ID NO:64, SEQ ID NO:66 and SEQ ID NO:68. In these examples, GST may be replaced by TrX.

Examples of TME2 mutated chimeric proteins according to the present invention are GST-DP-TME2_C731/C733A (SEQ ID NO:34) or TrX-DP-TME2_C731/C733A (SEQ ID NO:58), encoded, respectively by, for example, the oligonucleotide sequences SEQ ID NO:33 and SEQ ID NO:57.

The vector of the present invention can be obtained from any plasmid known to those skilled in the art of recombination DNA technology, for example an E. coli plasmid comprising (i) a region for replication and for maintenance of the plasmid in the host cell, and (ii) restriction sites for inserting the regions encoding the abovementioned chimeric proteins. The plasmid is chosen in particular by considering the host cell into which it will be introduced for coexpression.

The vector of the invention can be advantageously obtained from the plasmid pGEXKT (SEQ ID NO:23), or from the plasmid pET32a+ (SEQ ID NO:35). This is because these plasmids already comprise a sequence encoding a soluble protein (GST and TrX, respectively).

In the vector of the invention, the region for replication and for maintenance of the vector in the host cell is generally already present on the plasmid chosen for cloning the regions encoding the membrane proteins. If not, it can be inserted. These regions are known to those skilled in the art.

The promoters that precede the coding sequences for the chimeric proteins are DNA sequences recognized by RNA polymerase for initiation of transcription, which transcription subsequently takes place under the control of this enzyme. These promoters are known to those skilled in the art.

In order to obtain a vector according to the invention, from a plasmid chosen for cloning the membrane proteins and their coexpression, it is necessary to have available the nucleotides encoding said proteins, to which are attached, upstream in the 5′→3′ direction of each nucleotide and in this order, a nucleotide sequence encoding the DP dipeptide, and a nucleotide sequence encoding a soluble protein. Conventional recombinant DNA techniques, known to those skilled in the art, can be used. Briefly, restriction enzymes that make it possible to cleave the selected plasmid at given sites are used for inserting into the plasmid the regions encoding the membrane proteins to be coexpressed, each linked to a coding sequence for a soluble protein via a coding sequence for the DP dipeptide. Techniques that can be used are described, for example in [16]. A vector according to the present invention is then obtained.

By way of example, the vector of the present invention may be a vector of oligonucleotide sequence SEQ ID NO:61 or SEQ ID NO:62. The chimeric proteins coexpressed with these vectors are, respectively, GST-DP-TME2+TrX-DP-TME1 (SEQ ID NO:61) and GST-DP-TME2_C731/C733A+TrX-DP-TME1 (SEQ ID NO:62).

Also by way of example, the vector of the present invention may also be one of the following vectors encoding the following chimeric proteins:
vector SEQ ID NO:70 encoding the chimeric proteins
   GST-DP-TME2+TrX-DP-TME1_G354L (SEQ ID NO:31+SEQ ID NO:46);
vector SEQ ID NO:71 encoding the chimeric proteins
   GST-DP-TME2+TrX-DP-TME1_G358L (SEQ ID NO:31+SEQ ID NO:49);
vector SEQ ID NO:72 encoding the chimeric proteins
   GST-DP-TME2+TrX-DP-TME1_G354/358L (SEQ ID NO:31+SEQ ID NO:52);
vector SEQ ID NO:73 encoding the chimeric proteins
   TrX-DP-TME1_G354L+GST-DP-TME2_C731/733A (SEQ ID NO:46+SEQ ID NO:34);
vector SEQ ID NO:74 encoding the chimeric proteins
   TrX-DP-TME1_G358L+GST-DP-TME2_C731/733A (SEQ ID NO:49+SEQ ID NO:34); and
vector SEQ ID NO:75 encoding the chimeric proteins
   TrX-DP-TME1_G354/358L+GST-DP-TME2_C731/733A (SEQ ID NO:52+SEQ ID NO:34);

The other possible combinations with the various chimeric proteins presented above, for example with TrX-DP-TME2 or TrX-DP-TME2_C731/733A, are not explicitly set forth here in the interest of conciseness, but they are intended to be within the scope of this invention, as should be evident.

The vector of the present invention enables coexpression of the TME1 and TME2 membrane proteins of the HCV envelope, and to reproduce homo- and hetero-oligomeric forms of these proteins that would be present in the virus envelope.

The present invention also provides a prokaryotic cell transformed with an expression vector according to the invention. This transformed prokaryotic cell preferably allows the overexpression of the co-expressed membrane proteins encoded by the vector. Thus, any host cell 3 capable of expressing the expression vector of the present invention can be used, for example, E. coli, preferably the E. coli strain BL21(DE3)pLysS.

The present invention also provides a method for producing, by genetic recombination, hetero-oligomeric forms or a mixture of at least two membrane domains of the viral envelope proteins that interact in their native functional conformation in the virus envelope. The method may comprise the following steps:
transforming a host cell with a coexpression vector according to the invention,
culturing the transformed host cell under culture conditions wherein the vector nucleic acid is expressed resulting in production of the hetero-oligomeric forms or the mixture of the at least two membrane domains encoded by the vector, and
isolating the hetero-oligomers or the mixture from the above culture.
   The host cells and vectors that can be used are described above

This method, by virtue of the plasmid of the present invention, enables production of one or more hetero-oligomers or a mixture of at least two membrane domains of the viral envelope proteins, for example of the TME1 and TME2 membrane domains of the HCV envelope proteins. In fact, by exploiting the appropriate point mutations for impairing, or even eliminating, the interaction between the membrane domains produced, and therefore inhibiting or preventing formation of the hetero-oligomers, it is possible to obtain, using the present plasmid, a mixture of mutated peptides capable of being used in the various applications described below.

These hetero-oligomeric forms or the mixtures can form from the chimeric proteins, or from the membrane proteins produced, separated from their soluble protein and from the DP dipeptide. In fact, cleavage of the chimeric proteins produced can be carried out during the above isolation step, for example by means of formic acid, which cleaves the fusion protein at the DP dipeptide. The cleavage can be carried out, moreover, by any appropriate technique known to those skilled in the art for recovering an individual protein from a fusion protein.

In this respect, the present invention also provides a hetero-oligomer or a mixture of at least two membrane domains of the viral envelope proteins, which hetero-oligomer or mixture can be generated by the method of the invention, by use of the vector of the invention. In the case of HCV, it may, for example, be a hetero-oligomer or a mixture of at least one protein having a peptide sequence selected from the group SEQ ID NO:2, SEQ ID NO:10, SEQ ID NO:12 and SEQ ID NO:14 corresponding to the mutated or non-mutated TME1 peptide sequence; and of at least one protein having an amino acid sequence selected from the group SEQ ID NO:16 and SEQ ID NO:22 corresponding to the mutated or non-mutated TME2 amino acid sequences.

Also in this respect, since these proteins have different sizes and can therefore be separated, for example by electrophoresis, the present invention is also directed to one or other of these mutated proteins or the abovementioned mixture. It may, for example, be a protein having a peptide sequence selected from the group of sequences SEQ ID NO:10, SEQ ID NO:12, SEQ ID NO: 14, SEQ ID NO:65, SEQ ID NO:67 and SEQ ID NO:69 corresponding to the mutated TME1 amino acid sequence; and the amino acid sequences SEQ ID NO:16, SEQ ID NO:22, SEQ ID NO:34 and SEQ ID NO:58 corresponding to the mutated TME2 amino acid sequence. In fact, these proteins make it possible to prevent, or even eliminate, the formation of hetero-oligomers.

The proteins produced can be isolated from the host cells by conventional techniques known to those skilled in the art, provided that the technique used does not impair the oligomerization of the proteins produced. Techniques that can be used for this separation are, for example, electrophoretic and immunodetection techniques.

In this inventive approach, the inventors used a vector for expression as a fusion with GST to demonstrate homo-oligomeric forms of these chimeras. They then modified this system by replacing the GST with TrX, which made it possible to produce the same oligomers. They also demonstrated that the latter, despite their stability, are not maintained when certain mutations are present. Finally, the system was adapted to allow concomitant expression of the chimeras, which made it possible, entirely unexpectedly, (1) to reveal the existence of hetero-oligomers and, (2) to show that the mutations can limit their formation.

The existence of these associations or these mixtures of envelope proteins obtained using the vector of the present invention, and which appear to be essential to the formation of the virus, along with the means for producing them and of impairing them, provide a basis for novel therapeutics.

The first element of application is the vector for the coexpression of the membrane domains which is described here. Having been developed for expression in bacteria, the vector is very easy to use, enabling rapid testing of large numbers of compounds that can modulate the formation of the homo- and/or hetero-oligomeric forms identified here. This system is of great use for companies that seek to develop chemical agents against HCV.

For example, these virus envelope protein associations, or homo- and/or hetero-oligomers, that may or may not be mutated, obtained using the present vector, can be employed to produce monoclonal antibodies specific for these associations. Also, for example, by introducing mutations into just one or into several of the coexpressed proteins, prevent or impair these protein associations.

The present invention is also directed to the use of the membrane proteins, that may or may not be associated (hetero-oligomers and/or homo-oligomers), and that may or may not be mutated, or the use of a mixture of mutated proteins and of non-mutated proteins, for example mutated TME1 and TME2, non-mutated TME1 and TME2 or a mixture of TME1 and of TME2 in which just one of the two is mutated, for production of a pharmaceutical/medicinal product for use in the prophylaxis or treatment of a virus infection or a disease caused by the virus. An example is the treatment of hepatitis C disease.

The mutated forms of the membrane domains obtained using the plasmid of the present invention in fact have the ability to reduce the interactive forces of the domains. These peptides therefore constitute a novel type of inhibitor that can be used to compete with the wild-type forms of the envelope proteins, to impede the association thereof and thus to inhibit the virus production. The structure of these peptides can also serve as a basis for developing inhibitors.

The present invention also relates to the use of the associated membrane proteins (hetero-oligomers and/or homo-oligomers), that may or may not be mutated, for example TME1 and TME2 where just one of the two proteins is mutated, or both, in a screening method. In fact, as a result of the present invention, it is possible to test chemical or biological compounds, for example peptides, capable of interrupting the association of these envelope proteins. The chemical or biological compounds identified as interrupting these associations are potential candidate molecules for the development of novel active antiviral agents.

Vectors in accordance with the present invention and suitable for the abovementioned applications, in particular for hepatitis C, are, for example, the vectors having the sequence SEQ ID Nos. 61 and 62.

According to that which is known about other viruses similar to HCV, and their growth and replication cycles that are more thoroughly documented, it is believed that the envelope proteins adopt various intermediate association states. The reason for this is that the virus uses these various forms to allow each step of its cycle. For example, the E1E2 form present during the final phase of virus formation is not, however, that which allows the fusion/penetration into the host cell during infection. During this step, it is a homo-trimeric form of E1 that will be generated and used by the virus. It is assumed that other intermediate forms also exist, and this is what the inventors have discovered with the present invention.

It is known that the membrane domains of the two viral envelope proteins are responsible for a large part of this oligomerization phenomenon. What was not known up until the present invention and what the inventors therefore extended, is that, once the production of such proteins is made possible, it become practical to reproduce all the homo- and hetero-oligomerization states of the two viral envelope proteins.

The present invention is based on the premise that, if the formation of these complex forms can be disrupted, the formation of the envelope and therefore production of the virus are prevented. This provides a novel therapeutic approach which requires a tool that enables testing compounds capable of interfering with in the formation of these oligomers. This is what the present invention provides.

The present invention is based on the conception that for the successive phases of a virus's growth and replication cycle to occur, followed by viral fusion with the host cell's membrane, the two envelope proteins must associate in various states. These associations are generated and/or stabilized in part by the C-terminal membrane domains of the two proteins. If these associations are prevented, virus formation will be blocked at various stages, which will limit or will eliminate its infectivity. Compounds discovered and selected using the present invention should lead to the achievement of this aim.

The coexpression vector tool created by the present inventors is a system that enables precise and simple production of various complex forms that the membrane domains are capable of generating. The system utilizes bacteria and does not require a sophisticated expression system would be required to produce the complete envelope proteins (or their ectodomains).

The difficulty of producing membrane proteins in bacteria has been overcome here by fusing to these proteins, or their hydrophobic domains to the Asp-Pro dipeptide and to a soluble protein. It has not been commonplace to produce such hydrophobic domains and also to generate their various association states. This is because, while it is possible to synthesize chemically large quantities of peptides, the success of such an approach in the prior art was limited to hydrophilic peptides. Prior to the present invention, it was not possible to generate the corresponding hetero-oligomeric forms of hydrophobic peptides in vitro. In fact, the various hetero-oligomers, whether they comprise the complete viral envelope proteins or their C-terminal membrane domains, cannot be formed via independent chemical synthesis or biosynthesis of each of the constituents, followed simply by mixing them in solution. The present invention overcomes these obstacles and provides an in vivo approach for producing these peptides associated as hetero-oligomers by virtue of the novel plasmids. According to the present invention the formation of the complexes is exemplified as those generated by the C-terminal membrane domains TME1 and TME2, the spatial folding of which is much simpler than that of the full-length E1 and E2 proteins, and can therefore be carried out satisfactorily in a bacterial host cell system.

The examples below illustrate the application of the present invention. The inventors also discovered that, by introducing point mutations into one of the two membrane domains, they can limit the interaction between the two membrane proteins. This shows, first, that the these proteins are coproduced faithfully in bacteria and that the association states observed correspond to those which that occur intrinsically when these peptides interact in their native state.

The advantage for the pharmaceutical industry is evident: a sound and very inexpensive means for the high-throughput testing of chemical or biological agents potentially capable of preventing virus formation. The invention is of interest to companies that seek to develop inhibitors of these viruses (or membrane proteins).

The combined strategy developed in the present invention (coexpression, then point mutations) that encompasses both the association of membrane domains and its modulation by mutations, can be generalized not only to other pathologies caused by enveloped viruses, but also to any polytypic membrane protein involved in or responsible for a given pathology. One example is that of the ATP-binding cassette (ABC) transporters that play a major role in the multidrug resistance phenomenon and for which it becomes possible to search for specific inactivators using the present type of approach.

Other characteristics and advantages of the present invention will further emerge upon reading the description that follows, given by way of illustration, with reference to the figures and to the attached sequence listing.

### EXAMPLES

### Materials

The oligonucleotides used were obtained from the Laboratoires Eurobio, 07 Avenue de Scandinavie, 91953 Les Ulis Cedex B France, (see also world wide web address eurobio.fr). The vectors were prepared with the Qiaprep kit from Qiagen, 3 avenue du Canada, LP 809, 91974 Courtabœuf, Cedex, France (qiagen.com). The DNA sequences were sequenced with the ABI Prism® BigDye® Terminator Cycle kit from Applied Biosystems, 25 Avenue de la Baltique, B.P. 96, 91943 Courtabœuf, Cedex, France, home.appliedbiosystems.com. The E. coli strain BL21 Gold(DE3)pLysS and the QuickChange mutagenesis system were obtained from Stratagene, La Jolla, Calif., USA, stratagene.com. The DNA modification and restriction enzymes were obtained from New England Biolabs, UK, neb.com/neb. The protein electrophoresis and transfer apparatus is a MiniProtean 3®, the GS700 scanner coupled to the Molecular Analyst software and the molecular weight markers “Precision Protein standards” and “Kaleidoscope pre-stained standards” were obtained from the Bio-Rad Laboratoires, Division Bio-Recherche, 3 Boulevard Raymond Poincaré, 92430 Marnes la coquette, France,bio-rad.com. The plasmid pET32a+ was obtained from Novagen Inc, Madison, Wis. USA, novagen.com. The plasmid pGEXKT [18] was obtained from Prof. Dixon, Dept of Biological Chemistry, University of Michigan Ann Arbor, Mich. USA. The anti-GST antibody GST(Z-5):sc-459 was from Santa Cruz Biotechnology Inc., Santa Cruz, Calif. USA. The anti-TrX antibody Anti-Thio (#R920-25) and the vector pCRtopo2.1™ were from Invitrogen, SARL BP 96, CergyPontoise 95613.0 France. The ECL chemiluminescence kit and the LMW molecular weight markers were from Amersham Biosciences, Uppsala, Sweden. The peroxidase-conjugated goat anti-mouse antibody (#M32107) was from TEBU-bio SA, 39, Rue de Houdan, 78612 Le Perray en Yvelines Cedex France. Other products were obtained from Sigma, L'Isle d'Abeau Chesnes- B.P. 701, 38297 Saint-Quentin Fallavier, France, sigma-aldrich.com.

The following examples were carried out for the HCV TME1 and TME2 membrane domains of the E1 and E2 envelope proteins.

### Example 1

### Separate Expression of the GST-DP-TME1 and GST-DP-TME2 Chimeras

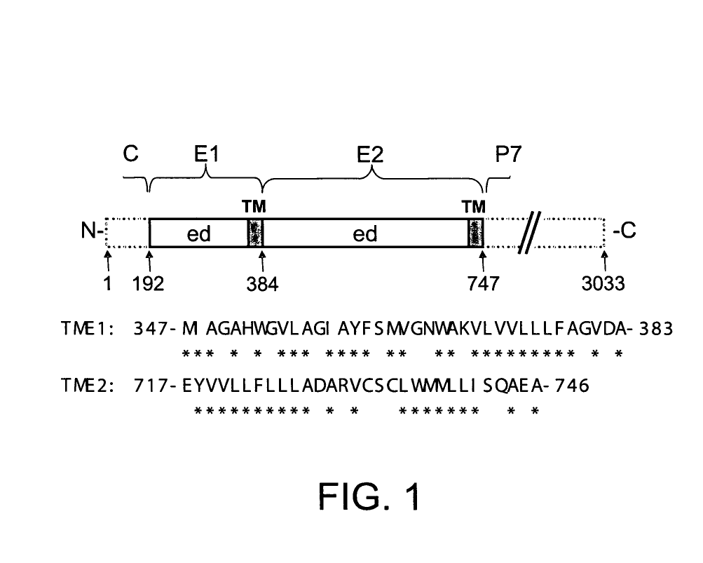

As indicated in FIG. 1, the membrane domains of the HCV envelope proteins TME1 and TME2 correspond respectively to segments of aa 347-383 (SEQ ID NO:2) and aa 717-746 (SEQ ID NO:16) of the polyprotein encoded by the viral RNA. Several different RNA sequences of HCV which produce an infectious phenotype exist. Those which were used to express TME1 and TME2 have the European Molecular Biology Laboratory (EMBL) public sequence library accession numbers, #D00831 and #M67463, respectively.

The DNA encoding TME1 and TME2 used in this example have the nucleotide sequence SEQ ID NO:1 and SEQ ID NO:15, respectively. These DNAs were synthesized de novo using the appropriate oligonucleotides. The codons were optimized for use in bacteria (Sharp et al. [26]). Each synthetic DNA was generated using a set of two long and overlapping oligonucleotides, OL11 (SEQ ID NO:76) and OL12 (SEQ ID NO:77) for TME1 and OL21 (SEQ ID NO:79) and OL22 (SEQ ID NO:80) for TME2.

These DNAs were subsequently amplified by PCR [27], by hybridization with two external oligonucleotides, OL17 (SEQ ID NO:78) and OL16 (SEQ ID NO:39) for TME1 and OL27 (SEQ ID NO:81) and OL26 (SEQ ID NO:40) for TME2, subsequently allowing subcloning into the plasmid pGEXKT.

The amplified DNAs were cloned into a bacterial plasmid pCRtopo2.1™ and sequenced. They were excised and then subcloned into the vector pGEXKT (SEQ ID NO:23) according to the protocol described in documents [17, 18], via the BamHI and EcoRI sites initially inserted 5′ and 3′ of the PCR fragments.

These membrane domains are produced as a C-terminal fusion with GST by integrating, between each domain and each soluble protein, a chemical cleavage site, DP, which makes it possible to reduce the intrinsic toxicity of the hydrophobic membrane protein.

The version of GST already present in the plasmid pGEXKT integrates at the end of its sequence a series of 5 glycine residues which confers a certain flexibility between the GST and the protein attached at this end.

The vectors pGEXKT-DP-TME1 (SEQ ID NO:26) and pGEXKT-DP-TME2 (SEQ ID NO:29) thus generated were incorporated into BL21 Gold(DE3)pLysS bacteria (B F⁻ dcm ompT hsdS(r_{B}⁻ m_{B}⁻) gal λ (DE3) [pLysS Cam^{r}]) to allow the expression of the GST-DP-TME1 and GST-DP-TME2 chimeras, the characteristics of which are noted in Table 1 below. In this table, the amino acids are indicated by single-letter code. The numbering of the sequences is carried out with respect to the proteins of origin, GST and viral polyprotein. That which refers to the membrane domains is shown in italics.

The expression of the chimeras is induced by isopropyl-1-thio-β-D-galactoside (IPTG). The host bacteria were modified to contain in the genome a copy of the gene encoding the T7 phage RNA polymerase, placed under the control of an isopropyl-1-thio-β-D-galactoside (IPTG)-inducible lacUV5 promoter. In this case, the bacteria were cultured at their optimum temperature of 37° C. or lower if necessary. The expression was induced by adding IPTG to the culture.

TABLE 1
GST-DP-TME1 and GST-DP-TME2 Chimeras

| | Chimera, | | | |
|---|---|---|---|---|
| Plasmid- | abbreviation, | | Size | Mass |
| vector | SEQ ID | Construct | (# aa's) | (Da) |
| pGEXKT | GST | — | 239 | 27469 |
| | SEQ ID NO: | | | |
| | 25 | | | |
| pGEXKT- | GST-DP- | ¹M-S²³³-DP-₃47M-A383 | 271 | 30718 |
| DP-TME1 | TME1, | | | |
| | GST- | | | |
| | DP-TME1 | | | |
| | SEQ ID | | | |
| | NO: 28 | | | |
| pGEXKT- | GST-DP- | ¹M-S²³³-DP-717E-A746 | 265 | 30403 |
| DP-TME2 | TME2, | | | |
| | GST-DP- | | | |
| | TME2 | | | |
| | SEQ ID | | | |
| | NO: 30 | | | |

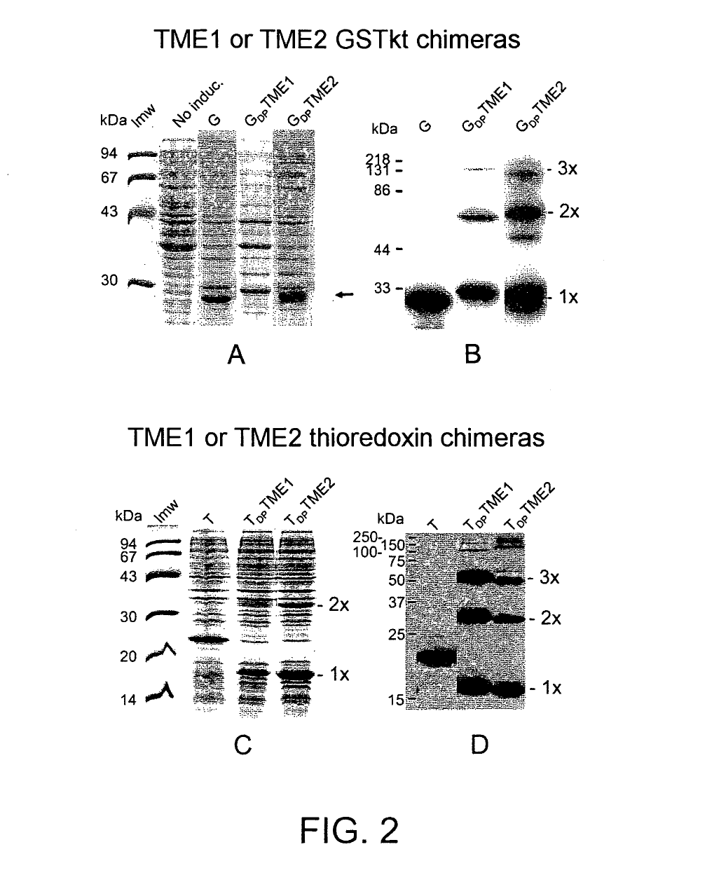

The proteins produced were subsequently separated by migration on a 12% PAGE gel carried out under “Laemmli”-type conditions, in the manner described in [19], and detected by Coomassie blue staining. Under these conditions, the results in the attached FIG. 2A were obtained in which, among the bacterial proteins, the GST-DP-TME1 and GST-DP-TME2 chimeras which were overproduced migrate at the expected size (˜30 kDa).

Unexpectedly, when the electrophoresis gels were treated by Western blotting to specifically reveal the GST chimeras with an antibody directed against GST (FIG. 2B), dimeric and trimeric forms of the GST-DP-TME1 and GST-DP-TME2 chimeras appeared.

The GST not fused to the membrane domains remained monomeric, which implies that the oligomerization is due to the presence of the hydrophobic regions. Similarly, the interactions that control the association of the membrane domains were sufficiently strong to at least partially withstand the very denaturing conditions to which the proteins are subjected during the preparation of the samples and their migration by SDS-PAGE (2% SDS, 4M urea, 0.7M of β-mercaptoethanol, migration, see description of FIG. 2).

These first results suggested, although did not convincingly prove, the oligomerization properties of the TME1 and TME2 membrane domains. This is because GST in solution is a dimer and this can promote the coming together of the domains. Similarly, TME2 contains the cysteines C731 and C733 for which the hydrophobic environment promotes oxidation, which can in turn promote aggregation of the domains. To evaluate these possibilities, the inventors transferred the constructs into a new plasmid to replace the GST with TrX in the chimeras.

### Example 2

### Expression of the Thioredoxin-DP-TME1 and Thioredoxin-DP-TME2 Chimeras

The replacement of GST with TrX in the chimeras was carried out using the expression plasmid pET32a+ (SEQ ID NO:35) In the latter, the sequence encoding TrX is inserted, in frame, as a short 3′ region added for detection and purification of the protein.

These elements were not used here, and insertion of the sequence encoding membrane domains was carried out just after the region encoding TrX, upstream of this additional portion.

The fragments (coding regions) to be inserted were generated by PCR using as template the vectors pGEXKT-DP-TME1 (SEQ ID NO:26) and pGEXKT-DP-TME2 (SEQ ID NO:29) and as primers the following sets of oligonucleotides:

TME1 and TME2, upstream oligonucleotide OL18(+):

| | |
|---|---|
| 5′-gtgatatctgatctgtctggtggtggt | (SEQ ID NO:38) |

TME1, downstream oligonucleotide OL16(−):

| | |
|---|---|
| 5′ gaattcctaagcttcagcctgag | SEQ ID NO:39 |

TME2, downstream oligonucleotide OL26(−):

| | |
|---|---|
| 5′ gaattcttaagcttcagcctgagagatcag | SEQ ID NO:40 |

The upstream oligonucleotide OL18 integrates an EcoRV site and hybridizes with segment from nucleotide 915 to nucleotide 932 of pGEXKT, corresponding to the terminal region of the gene encoding GST. The downstream oligonucleotide OL16 or OL26 is the same as that used for the cloning into pGEXKT. Using the pGEXKT-DP-TME1 and pGEXKT-DP-TME2 templates, each amplified fragment integrates the sequence SDLSGGGGGLVPRGS (SEQ ID NO:63), present at the C-terminus of the GST encoded by pGEXKT, followed by the DP site, followed by the membrane domain.

The insertion into the plasmid pET32a was via the MscI/EcoRV site in the 5′ position and EcoRI site in the 3′ position. This enabled inserting the amplified sequence at the end of, and in frame with the TrX coding sequence.

The vectors derived from these constructions are pET32a-TrX-DP-TME1 (SEQ ID NO:41) and pET32a-TrX-DP-TME2 (SEQ ID NO:53). The proteins produced from these vectors are TrX-DP-TME1 (SEQ ID NO:43) and TrX-DP-TME2 (SEQ ID NO:55). Their characteristics are summarized in Table 2 below. In this table, the amino acids are indicated with single-letter code. The numbering of the sequences is carried out with respect to the proteins of origin, GST and viral polyprotein. That which refers to the membrane domains is indicated in italics.

TABLE 2
Characteristics of the chimeric proteins from the vectors constructed

| | Chimera | | | |
|---|---|---|---|---|
| Plasmid- | <abbreviation> | | Size, | Mass |
| vector | (SEQ ID NO) | Construct | # aa's | (Da) |
| pET32a | thioredoxin, | ₁M-C₁₈₉ | 189 | 20397 |
| | <TrX> | | | |
| | (SEQ ID NO: 37) | | | |
| pET32a-DP- | TrX-DP-TME1, | ₁M-S₁₁₅-DP-T1 | 171 | 17796 |
| TME1 | <T_{DP}TME1> | | | |
| | (SEQ ID NO: 43) | | | |
| pET32a-DP- | TrX-DP-TME2, | ₁M-S₁₁₅-DP-T2 | 165 | 17481 |
| TME2 | <T_{DP}TME2> | | | |
| | (SEQ ID NO: 55) | | | |

The TrX-SDLSGGGGGLVPRGS-DP-(TME1) [SEQ ID NO:43] or TrX-SDLSGGGGGLVPRGS-DP-(TME2) [SEQ ID NO:55] (wherein SDLSGGGGGLVPRGS, as noted, is SEQ ID NO:63) are shorter than the protein encoded by the vector of origin because the insertion is carried out immediately after the TrX, which eliminates the sequence added downstream of the TrX which is of no interest here.

The expression of the TrX chimeras and the detection of the proteins produced were carried out as described in Example 1. The proteins produced were separated by 14% SDS-PAGE and then detected by Coomassie blue staining or by immunodetection.

FIG. 2C shows the presence among the bacterial proteins, of the TrX-DP-TME1 and TrX-DP-TME2 chimeras which were clearly overproduced and migrated at the expected size (˜18 kDa).

This result confirmed that the expression vector functioned with a protein other than GST.

The level of overexpression of the 2 proteins was such that their dimeric form (2× in FIG. 2C) was visible on the Coomassie blue-stained gel.

The immunodetection (Western blotting) (FIG. 2D) shows the presence of monomers (1×) and dimers (2×) but also, very clearly, the trimeric (3×) forms.

Since TrX does not form a dimer, these results clearly show that the oligomerization was due to the presence of the membrane domains. These results are the first experimental demonstration of the existence of oligomeric forms of TME1 and TME2.

### Example 3

### Expression of the GST and Thioredoxin Chimera Forms Mutated in the Membrane Domains

### Mutation C731A And C733A in TME2

As stated above, the mutation of the cysteine residues of TME2 was carried out to test their influence on the oligomerization of the GST-DP-TME2 chimeras.

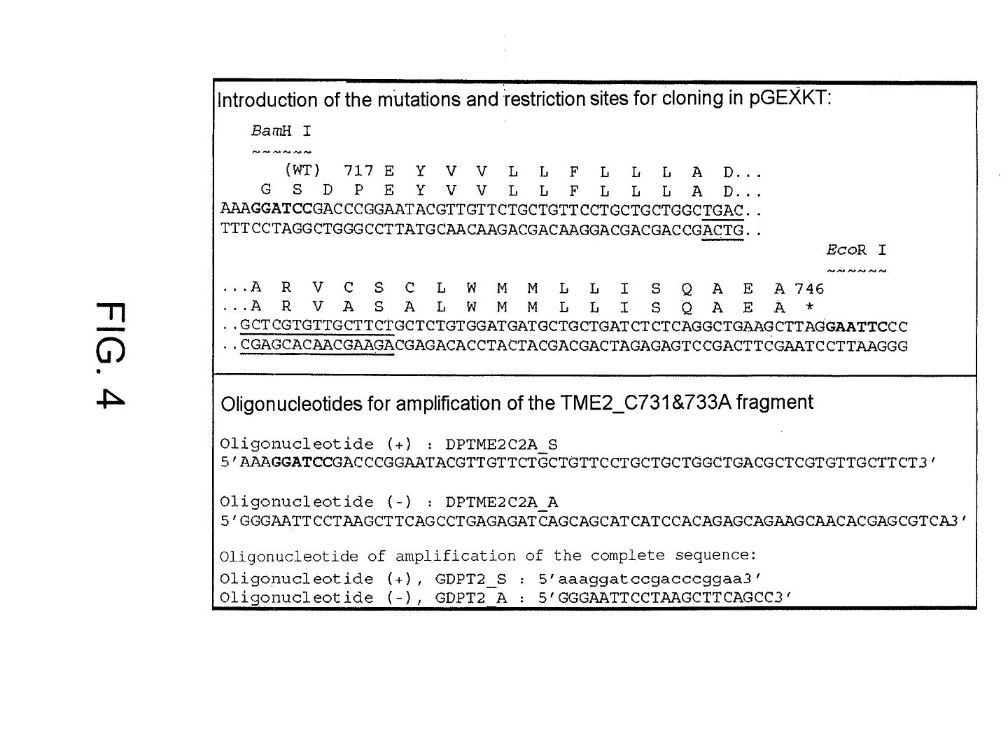

The mutagenesis was carried out by creating a new strand of DNA from long oligonucleotides as described in FIG. 4. The fragments generated were first cloned into the plasmid pGEXKT to create the vector pGEXKT-DP-TME2_C731/C733A (SEQ ID NO:32) allowing the expression of the GST-DP-TME2-C731/C733A chimera (SEQ ID NO:34), and then transferred into the plasmid pET32a with the strategy described in the preceding example so as to create the vector pET32a-DP-TME2_C731/C733A (SEQ ID NO:56) and generate the T_{DP}TME2-C731/C733A chimera (SEQ ID NO:58).

The DNA sequence encoding the C731A and C733A doubly mutated TME2 domain (SEQ ID NO:22) was synthesized de novo by PCR using the set of long oligonucleotides DPTME2C2A_S (SEQ ID NO:18) and DPTME2C2A_A (SEQ ID NO:19), which hybridize via their 3′ ends (underlined), while the external oligonucleotides GDPT2_S (SEQ ID NO:20) and GDPT2_A (SEQ ID NO:20) are used to facilitate the amplification after hybridization. The DNA generated is cleaved with BamHI and EcoRI and then inserted into the plasmid pGEXKT (SEQ ID NO:23). The sequence of the resulting vector pGEXKT-DP-TME2_C731/C733A (SEQ ID NO:32) is verified by sequencing.

The vectors resulting from the constructions were introduced into the BL21 Gold(DE3)pLysS bacteria and the expression was carried out as above.

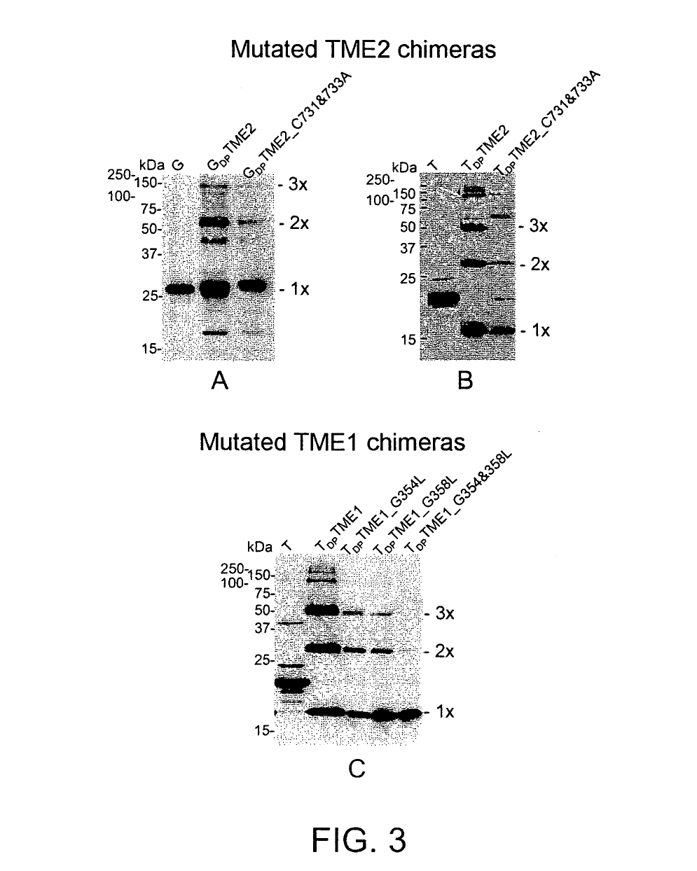

The proteins expressed were revealed by Coomassie blue staining (not shown) and Western blotting (FIG. 3).

FIG. 3A shows that the GST-DP-TME2-C731/C733A chimera was produced in quantities similar to those of its non-mutated form. However, the mutation very clearly decreased the level of dimer and reduced to trace amounts that of the trimer. The same result was obtained when TrX replaced the GST (FIG. 3B).

These results show first of all that the formation of the oligomers involving TME2 is not irreversible since a double mutation in the domain reduced the amount formed. Given that traces of oligomers were still visible on the gel, it is probable that the mutated domains also formed these oligomers; however, the double mutation reduces the strength of interaction sufficiently to prevent them from maintaining themselves under the denaturing conditions of the SDS-PAGE.

### Mutations G354L, G358L and G354/G358L in TME1

The inventors also tested the effect of mutations on the oligomerization of TME1. The choice of the residues to be mutated was made based on the studies by Op de Beeck et al., [11], showing that the addition of alanine residues in region 354-358 decreases the formation of the E1-E2 heterodimer. This region contains a “glycine” motif GXXXG. As was described by MacKenzie et al. [20], such a motif is critical for the association of membrane domains. This is because a membrane domain is generally an α-helix in which the two glycine residues of the motif, which are 4 residues apart, are spatially located below one another. Since the side chain of the glycine residues is limited to a hydrogen atom, the vacant space that results from the stacking of the two glycines can be filled with bulky hydrophobic residues, such as leucine, for example. This results in an embedding which strengthens the interaction between the domains. According to this principle, the inventors replaced the glycine residues at positions 354 and 358, independently and together, with a leucine so as to estimate their importance in this phenomenon.

The G354L, G358L and G354/G358L mutations were generated by the QuickChange™ system from Stratagene using as template the vector pET32A-TrX-DP-TME1. The mutations were not introduced into the GST chimeras.

The sets of oligonucleotides used to perform the G354L and G358L mutagenesis were:

T1G354L

(SEQ ID NO:3)

5′ GTAAGCGATACCAGCCAGAACCAGCCAGTGAGCACCAGCGAT-3′
T1G354Lc

(SEQ ID NO:4)

5′ ATCGCTGGTGCTCACTGGCTGGTTCTGGCTGGTATCGCTTAC 3′
T1G358L

(SEQ ID NO:5)

5′ CAACCATAGAGAAGTAAGCGATCAGAGCCAGAACACCCCAGTG
T1G358Lc

(SEQ ID NO;6)

5′ CACTGGGGTGTTCTGGCTCTGATCGCTTACTTCTCTATGGTTG 3′

The vectors generated were pET32A-TrX-DP-TME1_G354L (SEQ ID NO:44) and pET32A-TrX-DP-TME1_G358L (SEQ ID NO:47). They allow the expression of the TDPTME1-G354L (SEQ ID NO:46) and TDPTME1-G358L (SEQ ID NO:49) chimeras.

The double mutant was generated using as template the vector pET32A-TrX-DP-TME1_G354L and the following oligonucleotides (the bases underlined correspond to the codon is already mutated):

T1G2L

(SEQ: ID NO:7

5′ CAACCATAGAGAAGTAAGCGATCAGAGCCAGAACCAGCCAGTG 3′
T1G2Lc

(SEQ ID NO:8

5′ CACTGGCTGGTTCTGGCTCTGATCGCTTACTTCTCTAATGGTTG 3′

The vector created is pET32A-TrX-DP-TME1_G354/G358L (SEQ ID NO:50), generating the TDPTME1-G354/G358L chimera (SEQ ID NO:52).

As above, the vectors resulting from these constructions were introduced into the BL21 Gold(DE3)pLysS bacteria and the chimeras were expressed.

The proteins expressed were revealed by Western blotting. The results appear in FIG. 3C. By comparison within the non-modified domain, the replacement of glycine residues 354 or 358 with leucines clearly reduced the amount of trimer and also, though slightly less, the amount o of dimer. The simultaneous replacement of the two glycines resulted, on the other hand, in complete disappearance of the oligomers.

The glycine residues are therefore important for promoting the oligomerization of TME1, and this interaction is mainly due to the unit that they constitute since it is necessary to eliminate them together in order to obtain a complete effect.

These results add to the observations by Op De Beeck et al. [11], showing that the addition of alanine residues in region 354-358 which includes the two glycine residues (and not the replacement as is the case here) decreased the formation of the E1-E2 heterodimer. The expression of E1-E2 described by these authors had been carried out in a vaccinia system, quite similar to the natural conditions for expression of these complete proteins. (No system exists for expression of the complete virus, nor any system for its multiplication. The vaccinia system is one of the rare systems known to functionally express the complete E1 and E2 envelope proteins.)

It was therefore particularly advantageous to discover fact that, at least for the membrane domains of these proteins, the bacterial expression vector of the present invention enables reproduction of similar effects.

In this respect, the present vector appears to be as reliable as the vaccinia system, while being much simpler to use.

### Example 4

### Coexpression of the GST and Thioredoxin Chimeras

As already mentioned, no system exists for generating HCV, so it is therefore impossible to follow the steps that result in its formation. The few systems that make it possible to coexpress E1 and E2 remain difficult to use [28] and do not permit production of large amounts of proteins.

The present inventors developed a system for the coexpression of these domains showed herein that this system makes it possible to identify hetero-oligomeric forms of the chimeras such as they would exist during the formation of the virus.

In order to produce this system, the region of the vector pET32a-TrX-DP-TME1 containing the gene encoding the TrX-DP-TME1 chimera and its T7 promoter was first of all amplified by PCR (as described in Example 1) using the following set of oligonucleotides:

| | |
|---|---|
| PET998-AlwNI | |
| 5′-TTCAGTGGCTGTGCATGCAAGGAGATGGCG-3′ | (SEQ ID NO:59) |
| AST1-AlwNI | |
| 5′ TTCAGCCACTGCTAAGCGTCAACACCAGCG-3′ | (SEQ ID NO:60) |

The DNA cassette originating from the expression vector pET32a-TrX-DP-TME1 (SEQ ID NO:41), the construction of which is described in Example 2, comprises a T7 promoter followed by an open reading sequence containing, in frame, the gene encoding TrX followed by a DNA fragment encoding the Asp-Pro dipeptide, followed by the Met347-Ala383 region corresponding to the C-terminal membrane domain of E1. The corresponding chimeric protein is T_{DP}TME1 (SEQ ID NO:43, see Example 2).

The oligonucleotide PET998-AlwNI hybridized with segment 980-998 of pET32a, upstream of the T7 promoter for TrX. The oligonucleotide AST1-AlwNI hybridized with the 3′ region of the gene encoding TME1.

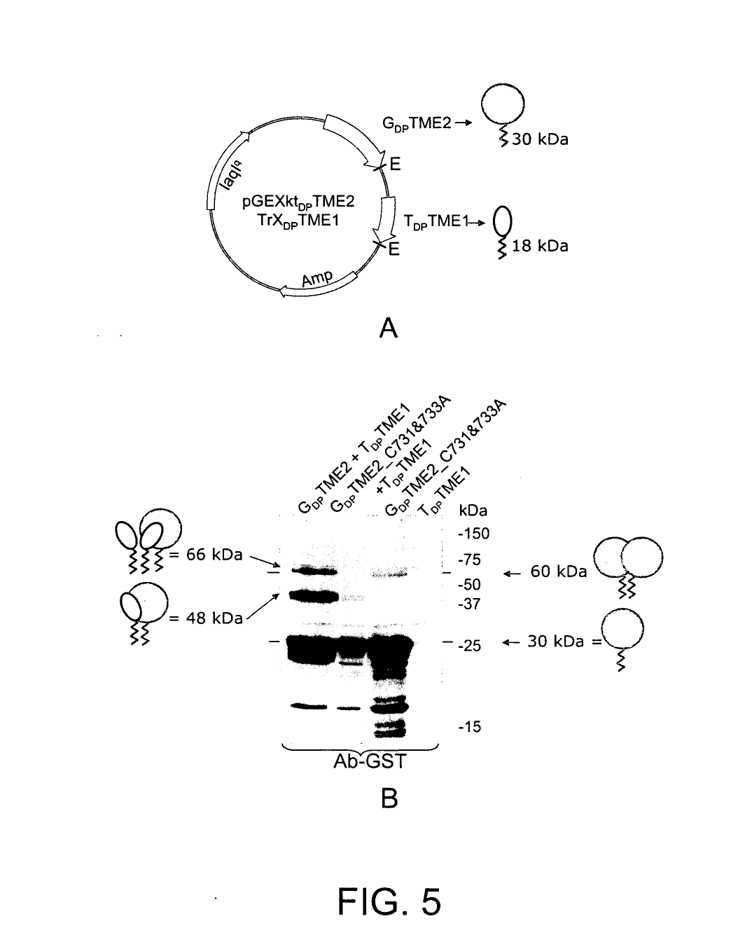

The PCR fragment was generated using these oligonucleotides and the vector pET32a-TrX-DP-TME1 (SEQ ID NO:41) as template. It was subsequently subcloned into the plasmid pCRtopo2.1™ and sequenced. It was subsequently excised from the plasmid pCRtopo2.1™ by restriction with the EcoRI enzyme, the two sites of which, present on the plasmid, are located a few bases before and after the subcloned fragment. The fragment thus excised was introduced into the unique EcoRI site in pGEXKT-DP-TME2 and pGEXKT-DP-TME2_C731/C733A, located downstream of the DNA encoding TME2 (cf., FIG. 5A for the position of the EcoRI site).

The vectors thus created are pGEXKT-DP-TME2+TrX-DP-TME1 (SEQ ID NO:61) and pGEXKT-DP-TME2_C731/C733A+TrX-DP-TME1 (SEQ ID NO:62). An example of a vector is illustrated in FIG. 5A. In this figure, the EcoRI site that was used to insert the cassette encoding the TrX-DP-TME1 chimera is indicated by the letter E. The chimeric proteins obtained are represented diagrammatically to the right of the vector, according to their size.

The reciprocal constructs producing the vectors pGEXKT-DP-TME1+TrX-DP-TME2 are not shown here. They give the same type of results as those described hereinafter.

The positive clones were cultured and induced as described in Example 1. Expression and association of the chimeras associate results in the appearance of various hetero-oligomers of the indicated molecular masses as summarized in Table 4

As above, the vectors resulting from these constructions were introduced into the BL21 Gold(DE3)pLysS bacteria and the chimeras were expressed.

TABLE 4
Possibilities of Association of Chimeras GST-DP-TME2
or GST-DP-TME2-C731/733A with Trx-DP-TME1, of
corresponding Molecular Mass

| | | MOLECULAR MASS (kDa) | | | |
|---|---|---|---|---|---|
| | | GST-DP-TME2 or GST-TME2-C731/733A | | | |
| | | Not | | | |
| | | expressed | Monomer | Dimer | Trimer |
| TrX-DP-TME1 | Not | — | 30 | 60 | 90 |
| | expressed | | | | |
| | Monomer | 18 | 48 | 78 | 108 |
| | Dimer | 36 | 66 | 96 | 126 |
| | Trimer | 54 | 84 | 114 | 144 |

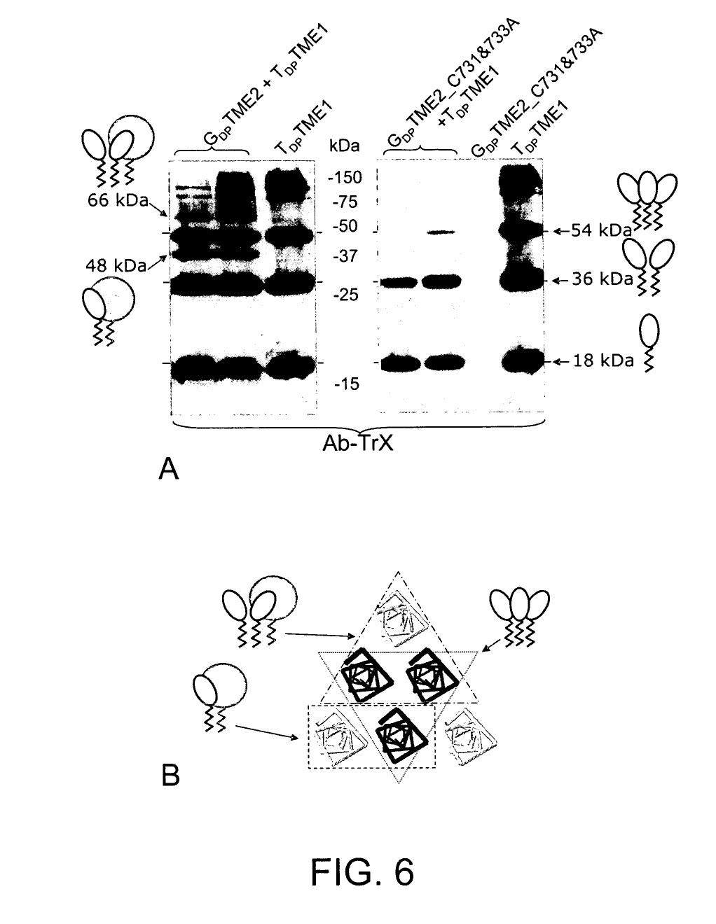

After expression, the bacteria were treated as described in Example 1. The various chimeras and also the oligomeric forms thereof were revealed by Western blotting and immunodetection using anti-GST (FIG. 5B) and anti-TrX (FIG. 6A) antibodies.

The coexpression assays were doubled in order to show the new species formed. In order to aid the reading of FIGS. 5 and 6, the GST-DP-TME2 and TrX-DP-TME1 (mutated or non-mutated) chimeric proteins were symbolized by icons, to show to what forms the homo- and hetero-oligomeric forms observed may correspond. The molecular weight markers used are the “Precision Protein standards”.

As illustrated in FIG. 5B, the visualization of the GST chimera products made it possible to detect the monomeric form GST-DP-TME2 migrating at 30 kDa. It was visible in all the lanes except lane 4 which had only the TrX-DP-TME1 chimera. In the lane of FIG. 5B, a band that was heavier than the monomeric form was visible. According to its migration its mass was compatible with 48 kDa, a mass that corresponds to the heterodimer

{GST-DP-TME2₁+TrX-DP-TME1₁}.

As can be seen in the lane 2, the amount of this heterodimeric species was greatly reduced when the C731/C733A double mutation was present in TME2. Finally, a larger form appeared as a band whose migration suggested that it could correspond to the heterotrimer {GST-DP-TME2₁+TrX-DP-TME1₂}. Despite the lower resolution in this region, the position on the gel of this heterotrimer of 66 kDa remained distinct from that of the GST-DP-TME2₂ homodimer (60 kDa), traces of which are visible in lane 3 where only the GST-DP-TME2-C731/C733A mutant is expressed. This heterotrimeric form is absent when TrX-DP-TME1 is coexpressed with the GST-DP-TME2-C731/C733A mutant, as can be seen in lane 2.

When the immunodetection was carried out with an anti-TrX antibody, the results presented in FIG. 6A showed that the coexpression of T-DP-TME1 with GST-DP-TME2 resulted first of all in the formation of the monomeric, dimeric and trimeric forms of TrX-DP-TME1 (see lanes 1, 2 and 3). Two new forms then appeared, which migrated on either side of the T-DP-TME1₃ homotrimer. The molecular masses of these proteins are compatible with those of the heterodimer {GST-DP-TME2₁+TrX-DP-TME1} and of the heterotrimer {GST-DP-TME21+TrX-DP-TME1₂}, which are 48 and 66 kDa, respectively.

This result therefore confirmed that obtained with the anti-GST antibody (lane 1 of FIG. 5B). When the coexpression assays were carried out with the TrX-DP-TME1 chimera and the GST-DP-TME2-C731/733A mutant (lanes 4 and 5 of FIG. 6A), it was clear that the hetero-oligomeric forms were no longer formed and, more unexpectedly, that the trimeric form TrX-DP-TME1₃ specifically was in low abundance.

These results show very clearly that the presence of the C731/C733A double mutation in TME2 weakened—to the point of making them disappear—the hetero-oligomeric forms and, even more notably, also contributed to decreasing the amount of the TrX-DP-TME13 trimers.

The first conclusion from the above experiments is that the coexpression of soluble proteins such as GST or TrX, fused to the TME1 and TME2 transmembrane domains of the HCV envelope proteins results in the formation of hetero-oligomeric species such as {GST-DP-TME2, +TrX-DP-TME1₁} and {GST-DP-TME21+TrX-DP-TME1₂}, which are sufficiently stable to withstand the denaturing conditions during the electrophoretic procedure. This is the first experimental demonstration of the ability of these membrane domains to associate with one another when they are expressed, independently or together. As these experiments were carried out in the absence of the ectodomain, which is the extra-membranous portion of the E1 and E2 proteins, the results showed the essential contribution of the membrane domains to this association phenomenon.

These results also showed quite clearly that the strength of the interactions that resulted in the formation of the homodimers was not equivalent for TME1 and TME2. This was particularly clear from the coexpression experiments which showed that the dimeric and trimeric species of TrX-DP-TME1 were always correctly formed despite the presence of the hetero-oligomers, whereas, in the case of GST-DP-TME2, the same species disappeared to the advantage of the hetero-oligomers.

This emphasizes the fact that TME1 and TME2 have intrinsically different oligomerization capacities, and provides information on their respective role during the virus formation. In fact, the most abundant/stable complexes that were formed during the coexpression and were still visible on SDS-PAGE gels were the (TrX-DP-TME1)₂ and (TrX-DP-TME1)₃ homo-oligomers and the {(GST-DP-TME2)₁+(TrX-DP-TME1)₁} and {(GST-DP-TME2)₁+(TrX-DP-TME1)₂} hetero-oligomers. These species withstood the denaturing conditions of the SDS-PAGE. It is therefore probable that they form a complex of a higher order under more physiological conditions.

The simplest organization of such a complex grouping of all the species observed corresponds to the condensation models represented diagrammatically in the center of FIG. 6B. The form thus generated consists, at its center, of a TrX-DP-TME1₃ trimer which is surrounded at the top by a GST-DP-TME2 monomer. This form could be the most “advanced” in terms of the structural organization of the virus, that which exists just before the fusion step. A similar organization was observed in the case of the tick-borne encephalitis virus [22-24].

### Example 5

### Validity of the System for the Coexpression of the Membrane Domains and Use for Discovering and Testing Compounds Capable of Impairing Stability of their Homo-Oligomeric and Hetero-Oligomeric Forms

The results described above showed that the inventors have invented a system for the coexpression, in bacteria, of the membrane domains of HCV envelope proteins. The heterodimeric forms that the inventors obtained correspond to those which were previously described when complete E1 and E2 proteins were coexpressed using a vaccinia system [11], which demonstrates that the vector of the present invention makes it possible to generate this form. It also enables generation of the {GST-DP-TME2₁+TrX-DP-TME1₂} heterotrimeric form which had not previously been observed.

The vector of the present invention therefore appears to be an excellent alternative for studying the interactions created by the membrane regions of envelope proteins.

Starting from the fact that the interaction of the envelope proteins involves the membrane regions and that the complexes that result therefrom are essential to the formation of the virus, it appears that this system makes it possible to test compounds capable of modulating the interactions used in these complexes and would be a major asset in finding agents that can combat this virus.

Once the present vector is made, it is extremely easy to use, highly economical, and makes possible the testing compounds on a scale compatible with that of combinatorial chemistry, for example.

Independently of this first approach used by the present inventors, they have also present herein a second approach that shows that it is possible to limit or eliminate the interaction of the membrane domains in the complexes that they can generate by introducing discrete mutations.

First, the double mutation of the glycine 354 and 358 residues to leucine residues eliminated the formation of the TME1 trimer, which would, according to the model of FIG. 5D, be one of the elements of the most mature form of the complex. Second, the double mutation of the cysteine 731 and 733 residues prevented the formation of the TME1 and TME2 hetero-oligomers, and also of the TME1 trimer.

These mutants serve as two examples of molecules that potentially compete with their wild-type form. In this respect, these molecules (peptides) are excellent candidates for combating the virus by impairing its formation and can be tested “as is” or in the form of derived products with a therapeutic aim.

### LIST OF REFERENCES

1. Ciccaglione A. R., Marcantonio C., Costantino A., Equestre M., Geraci A. and Rapicetta M. (2000) Virus Genes 21:223-226
2. Sisk W. P., Bradley J. D., Kingsley D., and Patterson T. A. (1992) Gene 112:57-162
3. Paulsen I. T., Sliwinski M. K., Nelissen B., Goffeau A., and Saler M. H. Jr. (1998) FEBS Lett 430:116-125
4. Decottignies A. and Goffeau A. (1997) Nat Genet. 15:137-145
5. Arechaga I., Miroux B., Karrasch S., Huijbregts R., de Kruijff B., Runswick M. J. and Walker J. E. (2000) FEBS Lett 482:215-219
6. Miroux B. and Walker J. E. (1996) J. Mol. Biol. 260:289-298
7. Mayo M. A., and Pringle C. R. (1998) J. Gen Virol. 79:649-657
8. Rosenberg, S. (2001) J Mol Biol 313:451-464.
9 Choo, Q. L., Kuo, G., Weiner, A. J., Overby, L. R., Bradley, D. W., and Houghton, M. (1989) Science 244:359-362.
10. Op de Beeck, A., Cocquerel, L., and Dubuisson, J (2001) J. Gen Virol 82:2589-2595.
11. Op de Beeck, A., Montserret, R., Duvet, S., Cooguerel, L., Cacan, R., Barberot, B., Le Maire, M., Penin, F., and Dubuisson, J. (2000) J. Biol Chem 275:31428-31437.
12. Ciccaglione, A. R., Marcantonio, C., Costantino, A., Equestre, M., Geraci, A., and Rapicetta, M. (1998) Virology 250:1-8.
13. Ciccaglione, A. R., Marcantonio, C., Costantino, A., Equestre, M., Geraci, A., and Rapicetta, M. (2000) Virus Genes 21:223-226.
14. Ciccaglione, A. R., Marcantonio, C., Equestre, M., Jones, I. M., and Rapicetta, M. (1998) Virus Res 55:157-165.
15. Sisk, W. P., Bradley, C. D., Kingsley, D., and Patterson, T. A. (1992) Gene 112:157-162.
16. J. Sambrook et al., Molecular Cloning, A Laboratory Manual, 2^{nd} edition, Cold Spring Harbor Laboratory Press, Cold Spring Harbor, N.Y., 1989.
17. Guan, K. L., and Dixon, J. E. (1991) Anal Biochem 192:262-267.
18. Hakes, D J, and Dixon, J E (1992) Anal Biochem 202:293-298.
19. Laemmli, U Y (1970) Nature 227:680-685.
20. MacKenzie, K. R., and Engelman, D. M. (1998) Proc Natl Acad Sci USA 95:3583-3590.
21. Dubuisson, J., Penin, F., and Mcradpour, D. (2002) Trends Cell Biol 12:517-523.
22. Ferlenghi, I., Clarke, M., Ruttan, T., Allison, S. L., Schalich, J., Heinz, F. X., Harrison, S. C., Rey, F. A., and Fuller, S. D. (2001) Mol Cell 7:593602.
24. Lorenz, I. C., Allison, S. L., Heinz, F. X., and Helenius, A. (2002) J Virol 76:5480-5491.
25. Stiasny, K., Allison, S. L., Schalich, J., and Heinz, F. X. (2002) J Virol 76:3784-3790.
26. Sharp, P. M., Cowe, E., Higgins, D. G., Shields, D. C., Wolfe, K. H. and Wright, F. (1988) Nucleic Acids Res. 16:8207-8211.
27. Mullis K. B., and Faloona F. A. (1987) Methods Enzymol 155:335-350.
28. Bartosch, B., Dubuisson, J., Cosset, Francois-Loic. (2003) J. Exp. Med. 197:633-642.

The references cited throughout this application are all incorporated by reference in their entirety, whether specifically incorporated or not.

1. A nucleic acid vector for the co-expression in a host cell of at least two membrane domains of viral envelope proteins that interact with one another when they are in a native, functional conformation in a virus envelope, said vector comprising:
(a) at least one region that controls replication and maintenance of said vector in the host cell;
(b) a first region consisting successively, in direction of translation of the vector, of
(i) a first promoter, followed by,
(ii) a first coding nucleotide sequence encoding a first chimeric protein and which, consists of, in the direction of translation of the vector,
(A) a first nucleotide sequence encoding a first soluble protein,
(B) a nucleotide sequence encoding an Asp-Pro dipeptide and
(C) a nucleotide sequence encoding one of said at least two membrane domains; and
(c) a second region consisting sequentially, in the direction of translation of the vector, of:
(i) a second promoter, followed by,
(ii) a second coding nucleotide sequence encoding a second chimeric protein, and which consists, in the direction of translation of the vector, of:
(A) a second nucleotide sequence encoding a second soluble protein,
(B) a nucleotide sequence encoding an Asp-Pro dipeptide, and
(C) a nucleotide sequence encoding the other of said at least two membrane domains.

2. A vector according to claim 1, in which the virus is one that is pathogenic for humans or for other mammals.

3. A vector according to claim 1, in which the first and the second regions are contiguous.

4. A vector according to claim 1, in which the first and second soluble proteins are glutathione S-transferase and/or thioredoxin.

5. A vector according to claim 1, in which the nucleotide sequence encoding the Asp-Pro dipeptide is GAC-CCG.

6. A vector according to claim 1, in which
(a) one of the two membrane domains has an amino acid sequence selected from the group of sequences SEQ ID NO:2; SEQ ID NO:10; SEQ ID NO:12 and SEQ ID NO:14, and
(b) the other membrane domain has an amino acid sequence selected from the group consisting of sequences SEQ ID NO:16 and SEQ ID NO:22.

7. A vector according to claim 6 in which,
(i) when one of the two domains has the sequence SEQ ID NO:2, the other domain is not the sequence SEQ ID NO:16, and
(ii) when one of the two domains has the sequence SEQ ID NO:16, the other domain is not the sequence SEQ ID NO:2.

8. A vector according to claim 1, which is obtained from the plasmid pEGEXKT having a sequence SEQ ID NO:23 or the plasmid pET32a+ having a sequence SEQ ID NO:35.

9. A vector according to claim 1, in which
(i) the sequence encoding one of said at least two membrane domains has a nucleotide sequence selected from the group consisting of the sequences SEQ ID NO:1, SEQ ID NO:9, SEQ ID NO:11 and SEQ ID NO:13, and
(ii) the sequence encoding the other of said at least two membrane domains has a nucleotide sequence selected from the group consisting of the sequences SEQ ID NO:15 and SEQ ID NO:17.

10. A vector according to claim 9, in which,
(i) when one of the two domains has the sequence SEQ ID NO:1, the other domain is does not have the sequence SEQ ID NO:15, and
(ii) when one of the two domains has the sequence SEQ ID NO:15, the other domain does not have the sequence SEQ ID NO:1.

11. An expression vector according to claim 1, in which
(a) the first encoded chimeric protein has a sequence selected from the group consisting of the sequences SEQ ID NO:28, SEQ ID NO:43, SEQ ID NO:46, SEQ ID NO:49 and SEQ ID NO:52, and
(b) the second encoded chimeric protein has a sequence selected from the group consisting of sequences SEQ ID NO:31, SEQ ID NO:34, SEQ ID NO:55 and SEQ ID NO:58.

12. A vector according to claim 11, in which,
(i) when the first encoded chimeric protein has the sequence SEQ ID NO:28, the second chimeric protein does not have the sequence SEQ ID NO:31, and
(ii) when the first encoded chimeric protein has the sequence SEQ ID NO:31, the second chimeric protein does not have the sequence SEQ ID NO:28.

13. An expression vector according to claim 1, which has a nucleotide sequence selected from the group consisting of the sequences SEQ ID NO:61; SEQ ID NO:62; SEQ ID NO:70; SEQ ID NO:71; SEQ ID NO:72; SEQ ID NO:73; SEQ ID NO:74 and SEQ ID NO:75.

14. A prokaryotic cell transformed with a vector according to claim 1.

15. A prokaryotic cell according to claim 14, which is an E. coli cell.

16. A method for recombinant production of a hetero-oligomer or a mixture of at least two membrane domains of viral envelope proteins that interact with one another in a native functional conformation in a virus envelope, comprising the following steps:
(a) transforming a host cell with a vector according to claim 1,
(b) culturing the transformed host cell under culture conditions wherein the vector nucleic acid is expressed, resulting in production of said hetero-oligomer or said mixture, and
(c) isolating said hetero-oligomer or said mixture from the culture of step (b).

17. A method according to claim 16, in which the host cell is an E. coli cell.

18. A method for recombinant production of a hetero-oligomer or a mixture comprising at least two membrane domains of viral envelope proteins that interact with one another in a native functional conformation in a virus envelope, comprising the following steps:
(a) transforming a host cell with a vector according to claim 6;
(b) culturing the transformed host cell under culture conditions wherein the vector nucleic acid is expressed resulting in production of said hetero-oligomer or said mixture; and
(c) isolating said hetero-oligomers or said mixture from the culture of step (b).

19. A method for recombinant production of a hetero-oligomer or a mixture comprising at least two membrane domains of viral envelope proteins that interact with one another in a native functional conformation in a virus envelope, comprising the following steps:
(a) transforming a host cell with a vector according to claim 8,
(b) culturing the transformed host cell under culture conditions wherein the vector nucleic acid is expressed resulting in production of said hetero-oligomer or said mixture; and
(c) isolating said hetero-oligomers or said mixture from the culture of step (b).

20. A method for recombinant production of a hetero-oligomer or a mixture comprising at least two membrane domains of viral envelope proteins that interact with one another in a native functional conformation in a virus envelope, comprising the following steps:
(a) transforming a host cell with a vector according to claim 9,
(b) culturing the transformed host cell under culture conditions wherein the vector nucleic acid is expressed resulting in production of said hetero-oligomer or said mixture; and
(c) isolating said hetero-oligomers or said mixture from the culture of step (b).

21. A method for recombinant production of a hetero-oligomer or a mixture comprising at least two membrane domains of viral envelope proteins that interact with one another in a native, functional conformation in a virus envelope, comprising the following steps:
(a) transforming a host cell with a vector according to claim 11,
(b) culturing the transformed host cell under culture conditions wherein the vector nucleic acid is expressed resulting in production of said hetero-oligomer or said mixture; and
(c) isolating said hetero-oligomers or said mixture from the culture of step (b).

22. A method for recombinant production of a hetero-oligomer or a mixture comprising at least two membrane domains of viral envelope proteins that interact with one another in a native functional conformation in a virus envelope, comprising the following steps:
(a) transforming a host cell with a vector according to claim 13,
(b) culturing the transformed host cell under culture conditions wherein the vector nucleic acid is expressed resulting in production of said hetero-oligomer or said mixture; and
(c) isolating said hetero-oligomers or said mixture from the culture of step (b).

23. A protein having an amino acid sequence selected from the group consisting of the sequences SEQ ID NO:10, SEQ ID NO:12, SEQ ID NO:14 and SEQ ID NO:22.

24. A hetero-oligomer or a mixture of at least a first and a second protein,
(i) the first protein having a sequence selected from the group consisting of the sequences SEQ ID NO:2, SEQ ID NO:10, SEQ ID NO:12, SEQ ID NO:14, SEQ ID NO:65, SEQ ID NO:67 and SEQ ID NO:69, and
(ii) the second protein having a sequence selected from the group consisting of sequences SEQ ID NO:16, SEQ ID NO:22, SEQ ID NO:34 and SEQ ID NO:58.

25. (canceled)

26. A method for treatment or prophylaxis of HCV infection or hepatitis C comprising administering to a subject in need thereof a protein according to claim 23 thereby resulting in said treatment or prophylaxis.

27. A method for treatment or prophylaxis of HCV infection of hepatitis C, comprising administering to a subject in need thereof a hetero-oligomer or mixture according to claim 24, thereby resulting in said treatment or prophylaxis.

28. A vector according to claim 2 wherein the virus is hepatitis C virus (HCV), or human immunodeficiency virus.


SEQUENCE LISTING (81 sequences)

SEQ ID NO: 1 - DNA, 111 bp, Hepatitis C virus
  DNA encoding the TME1 protein
atgatcgctggtgctcactggggtgttctggctggtatcgcttacttctctatggttggtaactgggctaaagttctggttgttctgctgctgttcgctggtgttgacgct

SEQ ID NO: 2 - protein, 37 aa, Hepatitis C virus
  TME1 protein
MIAGAHWGVLAGIAYFSMVGNWAKVLVVLLLFAGVDA

SEQ ID NO: 3 - DNA, 42 bp, Artificial sequence
  Artificial sequence descriptionβ oligonucleotide (+) for generating the TME1_G354L mutation
gtaagcgataccagccagaaccagccagtgagcaccagcgat

SEQ ID NO: 4 - DNA, 42 bp, Artificial sequence
  Artificial sequence descriptionβ oligonucleotide (-) for generating the TME1_G354L mutation
atcgctggtgctcactggctggttctggctggtatcgcttac

SEQ ID NO: 5 - DNA, 43 bp, Artificial sequence
  Artificial sequence descriptionβ oligonucleotide (+) for generating the TME1_G358L mutation
caaccatagagaagtaagcgatcagagccagaacaccccagtg

SEQ ID NO: 6 - DNA, 43 bp, Artificial sequence
  Artificial sequence descriptionβ oligonucleotide (-) for generating the TME1_G358L mutation
cactggggtgttctggctctgatcgcttacttctctatggttg

SEQ ID NO: 7 - DNA, 43 bp, Artificial sequence
  Artificial sequence descriptionβ oligonucleotide (+) for generating the TME1_G354&358L mutation
caaccatagagaagtaagcgatcagagccagaaccagccagtg

SEQ ID NO: 8 - DNA, 43 bp, Artificial sequence
  Artificial sequence descriptionβ oligonucleotide (-) for generating the TME1_G354&358L mutation
cactggctggttctggctctgatcgcttacttctctatggttg

SEQ ID NO: 9 - DNA, 111 bp, Artificial sequence
  Artificial sequence descriptionβ synthetic DNA encoding TME1_G354L
atgatcgctggtgctcactggctggttctggctggtatcgcttacttctctatggttggtaactgggctaaagttctggttgttctgctgctgttcgctggtgttgacgct

SEQ ID NO: 10 - protein, 37 aa, Artificial sequence
  Artificial sequence descriptionβ TME1_G354L protein
MIAGAHWLVLAGIAYFSMVGNWAKVLVVLLLFAGVDA

SEQ ID NO: 11 - DNA, 111 bp, Artificial sequence
  Artificial sequence descriptionβ synthetic DNA encoding TME1_G358L
atgatcgctggtgctcactggggtgttctggctctgatcgcttacttctctatggttggtaactgggctaaagttctggttgttctgctgctgttcgctggtgttgacgct

SEQ ID NO: 12 - protein, 37 aa, Artificial sequence
  Artificial sequence descriptionβ TME1_G358L protein
MIAGAHWGVLALIAYFSMVGNWAKVLVVLLLFAGVDA

SEQ ID NO: 13 - DNA, 111 bp, Artificial sequence
  Artificial sequence descriptionβ synthetic DNA encoding TME1_G354&G358L
atgatcgctggtgctcactggctggttctggctctgatcgcttacttctctatggttggtaactgggctaaagttctggttgttctgctgctgttcgctggtgttgacgct

SEQ ID NO: 14 - protein, 37 aa, Artificial sequence
  Artificial sequence descriptionβ TME1_G354&G358L protein
MIAGAHWLVLALIAYFSMVGNWAKVLVVLLLFAGVDA

SEQ ID NO: 15 - DNA, 90 bp, Hepatitis C virus
  Artificial sequence descriptionβ synthetic DNA encoding TME2
gaatacgttgttctgctgttcctgctgctggctgacgctcgtgtttgctcttgcctgtggatgatgctgctgatctctcaggctgaagct

SEQ ID NO: 16 - protein, 30 aa, Hepatitis C virus
  TME2 protein
EYVVLLFLLLADARVCSCLWMMLLISQAEA

SEQ ID NO: 17 - DNA, 90 bp, Artificial sequence
  Artificial sequence descriptionβ Synthetic DNA encoding TME2_C731&733A
gaatacgttgttctgctgttcctgctgctggctgacgctcgtgttgcttctgctctgtggatgatgctgctgatctctcaggctgaagct

SEQ ID NO: 18 - DNA, 66 bp, Artificial sequence
  Artificial sequence descriptionβ oligonucleotide (+) for creating the TME2_C731&733A fragment
aaaggatccgacccggaatacgttgttctgctgttcctgctgctggctgacgctcgtgttgcttct

SEQ ID NO: 19 - DNA, 69 bp, Artificial sequence
  Artificial sequence descriptionβ oligonucleotide (-) for creating the TME2_C731&733A fragment
gggaattcctaagcttcagcctgagagatcagcagcatcatccacagagcagaagcaacacgagcgtca

SEQ ID NO: 20 - DNA, 18 bp, Artificial sequence
  Artificial sequence descriptionβ oligonucleotide (+) for amplifying the TME2_C731&733A fragment
aaaggatccgacccggaa

SEQ ID NO: 21 - DNA, 21 bp, Artificial sequence
  Artificial sequence descriptionβ oligonucleotide (-) for amplifying the TME2_C731&733A fragment
gggaattcctaagcttcagcc

SEQ ID NO: 22 - protein, 30 aa, Artificial sequence
  Artificial sequence descriptionβ TME2_C731&733A protein
EYVVLLFLLLADARVASALWMMLLISQAEA

SEQ ID NO: 23 - DNA, 4969 bp, Artificial sequence
  Artificial sequence descriptionβ expression plasmid pGEXKT
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccccgggaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 24 - DNA, 717 bp, Artificial sequence
  Artificial sequence descriptionβ synthetic DNA encoding GST in the plasmid pGEXKT
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccccgggaattcatcgtgac

SEQ ID NO: 25 - protein, 239 aa, Artificial sequence
  Artificial sequence descriptionβ GST protein encoded in the plasmid pGEXKT
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLSGGGGGLVPRGSPGIHRD

SEQ ID NO: 26 - DNA, 5082 bp, Artificial sequence
  Artificial sequence descriptionβ vector pGEXKT-DP-TME1
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccgatcgctggtgctcactggggtgttctggctggtatcgcttacttctctatggttggtaactgggctaaagttctggttgttctgctgctgttcgctggtgttgacgcttaggaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 27 - DNA, 813 bp, Artificial sequence
  Artificial sequence descriptionβ synthetic DNA encoding the GST-DP-TME1 fusion protein in the vector pGEXKT-DP-TME1
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccgatcgctggtgctcactggggtgttctggctggtatcgcttacttctctatggttggtaactgggctaaagttctggttgttctgctgctgttcgctggtgttgacgct

SEQ ID NO: 28 - protein, 271 aa, Artificial sequence
  Artificial sequence descriptionβ GST-DP-TME1 protein encoded by the vector pGEXKT-DP-TME1
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLSGGGGGLVPRGSDPIAGAHWGVLAGIAYFSMVGNWAKVLVVLLLFAGVDA

SEQ ID NO: 29 - DNA, 5064 bp, Artificial sequence
  Artificial sequence descriptionβ vector pGEXKT-DP-TME2
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgtttgctcttgcctgtggatgatgctgctgatctctcaggctgaagcttaggaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 30 - DNA, 795 bp, Artificial sequence
  Artificial sequence descriptionβ synthetic DNA encodingt the GST-DP-TME2 fusion protein in the vector pGEXKT-DP-TME2
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgtttgctcttgcctgtggatgatgctgctgatctctcaggctgaagct

SEQ ID NO: 31 - protein, 265 aa, Artificial sequence
  Artificial sequence descriptionβ GST-DP-TME2 protein encoded by the vector pGEXKT-DP-TME2
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLSGGGGGLVPRGSDPEYVVLLFLLLADARVCSCLWMMLLISQAEA

SEQ ID NO: 32 - DNA, 5064 bp, Artificial sequence
  Artificial sequence descriptionβ vector pGEXKT-DP-TME2_C731&C733A
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgttgcttctgctctgtggatgatgctgctgatctctcaggctgaagcttaggaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 33 - DNA, 795 bp, Artificial sequence
  Artificial sequence descriptionβ synthetic DNA encoding the GST-DP-TME2_C731&C733A fusion protein in the vector pGEXKT-DP-TME2
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgttgcttctgctctgtggatgatgctgctgatctctcaggctgaagct

SEQ ID NO: 34 - protein, 265 aa, Artificial sequence
  Artificial sequence descriptionβ GST-DP-TME2_C731&C733A protein encoded by the vector pGEXKT-DP-TME2
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLSGGGGGLVPRGSDPEYVVLLFLLLADARVASALWMMLLISQAEA

SEQ ID NO: 35 - DNA, 11800 bp, Artificial sequence
  Artificial sequence descriptionβ expression plasmid pET32a+
atccggatatagttcctcctttcagcaaaaaacccctcaagacccgtttagaggccccaaggggttatgctagttattgctcagcggtggcagcagccaataggcctatatcaaggaggaaagtcgttttttggggagttctgggcaaatctccggggttccccaatacgatcaataacgagtcgccaccgtcgtcggttctcagcttcctttcgggctttgttagcagccggatctcagtggtggtggtggtggtgctcgagtgcggccgcaagcttgtcgacggagctcgaattcggagagtcgaaggaaagcccgaaacaatcgtcggcctagagtcaccaccaccaccaccacgagctcacgccggcgttcgaacagctgcctcgagcttaagccttccgatatcagccatggccttgtcgtcgtcgtcggtacccagatctgggctgtccatgtgctggcgttcgaatttagcagcagcggtttctttcataccaaggctatagtcggtaccggaacagcagcagcagccatgggtctagacccgacaggtacacgaccgcaagcttaaatcgtcgtcgccaaagaaagtatggtgaaccgcgtggcaccagaccagaagaatgatgatgatgatggtgcatatggccagaaccagaaccggccaggttagcgtcgaggaactctttcaactgaccttggcgcaccgtggtctggtcttcttactactactactaccacgtataccggtcttggtcttggccggtccaatcgcagctccttgagaaagttgactgctttagacagtgcacccactttggttgccgccacttcaccgtttttgaacagcagcagagtcgggataccacggatgccatatttcggcgcagtgccagggaaatctgtcacgtgggtgaaaccaacggcggtgaagtggcaaaaacttgtcgtcgtctcagccctatggtgcctacggtataaagccgcgtcacggtccgttttgatcgatgttcagttttgcaacggtcagtttgccctgatattcgtcagcgatttcatccagaatcggggcgatcattttgcacggaccgcaccaccaaaactagctacaagtcaaaacgttgccagtcaaacgggactataagcagtcgctaaagtaggtcttagccccgctagtaaaacgtgcctggcgtggtgtctgcccagaaatcgacgaggatcgccccgtccgctttgagtacatccgtgtcaaaactgtcgtcagtcaggtgaataattttatcgctcatatgtatatagacgggtctttagctgctcctagcggggcaggcgaaactcatgtaggcacagttttgacagcagtcagtccacttattaaaatagcgagtatacatatactccttcttaaagttaaacaaaattatttctagaggggaattgttatccgctcacaattcccctatagtgagtcgtattaatttcgcgggatcgagatcggaggaagaatttcaatttgttttaataaagatctccccttaacaataggcgagtgttaaggggatatcactcagcataattaaagcgccctagctctagcatctcgatcctctacgccggacgcatcgtggccggcatcaccggcgccacaggtgcggttgctggcgcctatatcgccgacatcaccgatggggaagatctagagctaggagatgcggcctgcgtagcaccggccgtagtggccgcggtgtccacgccaacgaccgcggatatagcggctgtagtggctaccccttctaggggctcgccacttcgggctcatgagcgcttgtttcggcgtgggtatggtggcaggccccgtggccgggggactgttgggcgccatctccttgcatgcacccccgagcggtgaagcccgagtactcgcgaacaaagccgcacccataccaccgtccggggcaccggccccctgacaacccgcggtagaggaacgtacgtggattccttgcggcggcggtgctcaacggcctcaacctactactgggctgcttcctaatgcaggagtcgcataagggagagcgtcgagatcccggacaccattaaggaacgccgccgccacgagttgccggagttggatgatgacccgacgaaggattacgtcctcagcgtattccctctcgcagctctagggcctgtggtacgaatggcgcaaaacctttcgcggtatggcatgatagcgcccggaagagagtcaattcagggtggtgaatgtgaaaccagtaacgttatacgatgtcgcagcttaccgcgttttggaaagcgccataccgtactatcgcgggccttctctcagttaagtcccaccacttacactttggtcattgcaatatgctacagcgtgagtatgccggtgtctcttatcagaccgtttcccgcgtggtgaaccaggccagccacgtttctgcgaaaacgcgggaaaaagtggaagcggcgatggcggctcatacggccacagagaatagtctggcaaagggcgcaccacttggtccggtcggtgcaaagacgcttttgcgccctttttcaccttcgccgctaccgccagctgaattacattcccaaccgcgtggcacaacaactggcgggcaaacagtcgttgctgattggcgttgccacctccagtctggccctgcacgcgccgtctcgacttaatgtaagggttggcgcaccgtgttgttgaccgcccgtttgtcagcaacgactaaccgcaacggtggaggtcagaccgggacgtgcgcggcaggcaaattgtcgcggcgattaaatctcgcgccgatcaactgggtgccagcgtggtggtgtcgatggtagaacgaagcggcgtcgaagcctgtaaagcggcgcgtttaacagcgccgctaatttagagcgcggctagttgacccacggtcgcaccaccacagctaccatcttgcttcgccgcagcttcggacatttcgccgcgtgcacaatcttctcgcgcaacgcgtcagtgggctgatcattaactatccgctggatgaccaggatgccattgctgtggaagctgcctgcactaatgttccacgtgttagaagagcgcgttgcgcagtcacccgactagtaattgataggcgacctactggtcctacggtaacgacaccttcgacggacgtgattacaagcggcgttatttcttgatgtctctgaccagacacccatcaacagtattattttctcccatgaagacggtacgcgactgggcgtggagcatctggtcgcattgccgcaataaagaactacagagactggtctgtgggtagttgtcataataaaagagggtacttctgccatgcgctgacccgcacctcgtagaccagcgtaagggtcaccagcaaatcgcgctgttagcgggcccattaagttctgtctcggcgcgtctgcgtctggctggctggcataaatatctcactcgcaatcaaattcccagtggtcgtttagcgcgacaatcgcccgggtaattcaagacagagccgcgcagacgcagaccgaccgaccgtatttatagagtgagcgttagtttaacagccgatagcggaacgggaaggcgactggagtgccatgtccggttttcaacaaaccatgcaaatgctgaatgagggcatcgttcccactgcgatgctgggtcggctatcgccttgcccttccgctgacctcacggtacaggccaaaagttgtttggtacgtttacgacttactcccgtagcaagggtgacgctacgaccttgccaacgatcagatggcgctgggcgcaatgcgcgccattaccgagtccgggctgcgcgttggtgcggacatctcggtagtgggatacgacgataccgaaacggttgctagtctaccgcgacccgcgttacgcgcggtaatggctcaggcccgacgcgcaaccacgcctgtagagccatcaccctatgctgctatggctagacagctcatgttatatcccgccgttaaccaccatcaaacaggattttcgcctgctggggcaaaccagcgtggaccgcttgctgcaactctctcagggctctgtcgagtacaatatagggcggcaattggtggtagtttgtcctaaaagcggacgaccccgtttggtcgcacctggcgaacgacgttgagagagtcccgcaggcggtgaagggcaatcagctgttgcccgtctcactggtgaaaagaaaaaccaccctggcgcccaatacgcaaaccgcctctccccgcgcgttggccggtccgccacttcccgttagtcgacaacgggcagagtgaccacttttctttttggtgggaccgcgggttatgcgtttggcggagaggggcgcgcaaccggcattcattaatgcagctggcacgacaggtttcccgactggaaagcgggcagtgagcgcaacgcaattaatgtaagttagctcactcattaggcaccgggattaagtaattacgtcgaccgtgctgtccaaagggctgacctttcgcccgtcactcgcgttgcgttaattacattcaatcgagtgagtaatccgtggccctactcgaccgatgcccttgagagccttcaacccagtcagctccttccggtgggcgcggggcatgactatcgtcgccgcacttatgactgtcttctttatcatgagctggctacgggaactctcggaagttgggtcagtcgaggaaggccacccgcgccccgtactgatagcagcggcgtgaatactgacagaagaaatagtagcaactcgtaggacaggtgccggcagcgctctgggtcattttcggcgaggaccgctttcgctggagcgcgacgatgatcggcctgtcgcttgcggtattccgttgagcatcctgtccacggccgtcgcgagacccagtaaaagccgctcctggcgaaagcgacctcgcgctgctactagccggacagcgaacgccataagggaatcttgcacgccctcgctcaagccttcgtcactggtcccgccaccaaacgtttcggcgagaagcaggccattatcgccggcatggcggccccacgggccttagaacgtgcgggagcgagttcggaagcagtgaccagggcggtggtttgcaaagccgctcttcgtccggtaatagcggccgtaccgccggggtgccctgcgcatgatcgtgctcctgtcgttgaggacccggctaggctggcggggttgccttactggttagcagaatgaatcaccgatacgcgagcgaacgtgaagacgcgtactagcacgaggacagcaactcctgggccgatccgaccgccccaacggaatgaccaatcgtcttacttagtggctatgcgctcgcttgcacttccgactgctgctgcaaaacgtctgcgacctgagcaacaacatgaatggtcttcggtttccgtgtttcgtaaagtctggaaacgcggaagtcagcgccctgcgctgacgacgacgttttgcagacgctggactcgttgttgtacttaccagaagccaaaggcacaaagcatttcagacctttgcgccttcagtcgcgggacgaccattatgttccggatctgcatcgcaggatgctgctggctaccctgtggaacacctacatctgtattaacgaagcgctggcattgaccctgagtgattttggtaatacaaggcctagacgtagcgtcctacgacgaccgatgggacaccttgtggatgtagacataattgcttcgcgaccgtaactgggactcactaaattctctggtcccgccgcatccataccgccagttgtttaccctcacaacgttccagtaaccgggcatgttcatcatcagtaacccgtatcgtgagcatcctaagagaccagggcggcgtaggtatggcggtcaacaaatgggagtgttgcaaggtcattggcccgtacaagtagtagtcattgggcatagcactcgtaggactctcgtttcatcggtatcattacccccatgaacagaaatcccccttacacggaggcatcagtgaccaaacaggaaaaaaccgcccttaacatggcccgcgagagcaaagtagccatagtaatgggggtacttgtctttagggggaatgtgcctccgtagtcactggtttgtccttttttggcgggaattgtaccgggcgtttatcagaagccagacattaacgcttctggagaaactcaacgagctggacgcggatgaacaggcagacatctgtgaatcgcttcacgaccacgctgatgaaatagtcttcggtctgtaattgcgaagacctctttgagttgctcgacctgcgcctacttgtccgtctgtagacacttagcgaagtgctggtgcgactacagctttaccgcagctgcctcgcgcgtttcggtgatgacggtgaaaacctctgacacatgcagctcccggagacggtcacagcttgtctgtaagcggatgctcgaaatggcgtcgacggagcgcgcaaagccactactgccacttttggagactgtgtacgtcgagggcctctgccagtgtcgaacagacattcgcctacgcgggagcagacaagcccgtcagggcgcgtcagcgggtgttggcgggtgtcggggcgcagccatgacccagtcacgtagcgatagcggagtgtatactggcgccctcgtctgttcgggcagtcccgcgcagtcgcccacaaccgcccacagccccgcgtcggtactgggtcagtgcatcgctatcgcctcacatatgaccgttaactatgcggcatcagagcagattgtactgagagtgcaccatatatgcggtgtgaaataccgcacagatgcgtaaggagaaaataccgcatcaggcgcaattgatacgccgtagtctcgtctaacatgactctcacgtggtatatacgccacactttatggcgtgtctacgcattcctcttttatggcgtagtccgcgtcttccgcttcctcgctcactgactcgctgcgctcggtcgttcggctgcggcgagcggtatcagctcactcaaaggcggtaatacggttatccacagaatagaaggcgaaggagcgagtgactgagcgacgcgagccagcaagccgacgccgctcgccatagtcgagtgagtttccgccattatgccaataggtgtcttacaggggataacgcaggaaagaacatgtgagcaaaaggccagcaaaaggccaggaaccgtaaaaaggccgcgttgctggcgtttttccataggctccgcccgtcccctattgcgtcctttcttgtacactcgttttccggtcgttttccggtccttggcatttttccggcgcaacgaccgcaaaaaggtatccgaggcgggccctgacgagcatcacaaaaatcgacgctcaagtcagaggtggcgaaacccgacaggactataaagataccaggcgtttccccctggaagctccctcgtggggactgctcgtagtgtttttagctgcgagttcagtctccaccgctttgggctgtcctgatatttctatggtccgcaaagggggaccttcgagggagcaccgctctcctgttccgaccctgccgcttaccggatacctgtccgcctttctcccttcgggaagcgtggcgctttctcatagctcacgctgtaggtatctcagcgagaggacaaggctgggacggcgaatggcctatggacaggcggaaagagggaagcccttcgcaccgcgaaagagtatcgagtgcgacatccatagagtgttcggtgtaggtcgttcgctccaagctgggctgtgtgcacgaaccccccgttcagcccgaccgctgcgccttatccggtaactatcgtcttgagtccaacaagccacatccagcaagcgaggttcgacccgacacacgtgcttggggggcaagtcgggctggcgacgcggaataggccattgatagcagaactcaggttcccggtaagacacgacttatcgccactggcagcagccactggtaacaggattagcagagcgaggtatgtaggcggtgctacagagttcttgaagtggtgggggccattctgtgctgaatagcggtgaccgtcgtcggtgaccattgtcctaatcgtctcgctccatacatccgccacgatgtctcaagaacttcaccacccctaactacggctacactagaaggacagtatttggtatctgcgctctgctgaagccagttaccttcggaaaaagagttggtagctcttgatccggcaaacggattgatgccgatgtgatcttcctgtcataaaccatagacgcgagacgacttcggtcaatggaagcctttttctcaaccatcgagaactaggccgtttgaaaccaccgctggtagcggtggtttttttgtttgcaagcagcagattacgcgcagaaaaaaaggatctcaagaagatcctttgatcttttctacggggtctttggtggcgaccatcgccaccaaaaaaacaaacgttcgtcgtctaatgcgcgtctttttttcctagagttcttctaggaaactagaaaagatgccccagtgacgctcagtggaacgaaaactcacgttaagggattttggtcatgagattatcaaaaaggatcttcacctagatccttttaaattaaaaatgaagttttactgcgagtcaccttgcttttgagtgcaattccctaaaaccagtactctaatagtttttcctagaagtggatctaggaaaatttaatttttacttcaaaaaaatcaatctaaagtatatatgagtaaacttggtctgacagttaccaatgcttaatcagtgaggcacctatctcagcgatctgtctatttcgttcatccatttagttagatttcatatatactcatttgaaccagactgtcaatggttacgaattagtcactccgtggatagagtcgctagacagataaagcaagtaggttagttgcctgactccccgtcgtgtagataactacgatacgggagggcttaccatctggccccagtgctgcaatgataccgcgagacccacgctcaccggcatcaacggactgaggggcagcacatctattgatgctatgccctcccgaatggtagaccggggtcacgacgttactatggcgctctgggtgcgagtggccgtccagatttatcagcaataaaccagccagccggaagggccgagcgcagaagtggtcctgcaactttatccgcctccatccagtctattaattgttgccggaggtctaaatagtcgttatttggtcggtcggccttcccggctcgcgtcttcaccaggacgttgaaataggcggaggtaggtcagataattaacaacggccgaagctagagtaagtagttcgccagttaatagtttgcgcaacgttgttgccattgctgcaggcatcgtggtgtcacgctcgtcgtttggtatggcttcatcttcgatctcattcatcaagcggtcaattatcaaacgcgttgcaacaacggtaacgacgtccgtagcaccacagtgcgagcagcaaaccataccgaagtatcagctccggttcccaacgatcaaggcgagttacatgatcccccatgttgtgcaaaaaagcggttagctccttcggtcctccgatcgttgtcagaagtaaagtcgaggccaagggttgctagttccgctcaatgtactagggggtacaacacgttttttcgccaatcgaggaagccaggaggctagcaacagtcttcattgttggccgcagtgttatcactcatggttatggcagcactgcataattctcttactgtcatgccatccgtaagatgcttttctgtgactggtgagtactcacaaccggcgtcacaatagtgagtaccaataccgtcgtgacgtattaagagaatgacagtacggtaggcattctacgaaaagacactgaccactcatgagtaccaagtcattctgagaatagtgtatgcggcgaccgagttgctcttgcccggcgtcaatacgggataataccgcgccacatagcagaactttaaaagtgctggttcagtaagactcttatcacatacgccgctggctcaacgagaacgggccgcagttatgccctattatggcgcggtgtatcgtcttgaaattttcacgtcatcattggaaaacgttcttcggggcgaaaactctcaaggatcttaccgctgttgagatccagttcgatgtaacccactcgtgcacccaactgatcttcagtagtaaccttttgcaagaagccccgcttttgagagttcctagaatggcgacaactctaggtcaagctacattgggtgagcacgtgggttgactagaagagcatcttttactttcaccagcgtttctgggtgagcaaaaacaggaaggcaaaatgccgcaaaaaagggaataagggcgacacggaaatgttgaatactctcgtagaaaatgaaagtggtcgcaaagacccactcgtttttgtccttccgttttacggcgttttttcccttattcccgctgtgcctttacaacttatgagatactcttcctttttcaatattattgaagcatttatcagggttattgtctcatgagcggatacatatttgaatgtatttagaaaaataaacaaataggggtatgagaaggaaaaagttataataacttcgtaaatagtcccaataacagagtactcgcctatgtataaacttacataaatctttttatttgtttatccccttccgcgcacatttccccgaaaagtgccacctgaaattgtaaacgttaatattttgttaaaattcgcgttaaatttttgttaaatcagctcattttttaaaaggcgcgtgtaaaggggcttttcacggtggactttaacatttgcaattataaaacaattttaagcgcaatttaaaaacaatttagtcgagtaaaaaattccaataggccgaaatcggcaaaatcccttataaatcaaaagaatagaccgagatagggttgagtgttgttccagtttggaacaagagtccactattaaagggttatccggctttagccgttttagggaatatttagttttcttatctggctctatcccaactcacaacaaggtcaaaccttgttctcaggtgataatttcaacgtggactccaacgtcaaagggcgaaaaaccgtctatcagggcgatggcccactacgtgaaccatcaccctaatcaagttttttggggtcgaggtgccttgcacctgaggttgcagtttcccgctttttggcagatagtcccgctaccgggtgatgcacttggtagtgggattagttcaaaaaaccccagctccacgggtaaagcactaaatcggaaccctaaagggagcccccgatttagagcttgacggggaaagccggcgaacgtggcgagaaaggaagggaagaaagcgaaaggcatttcgtgatttagccttgggatttccctcgggggctaaatctcgaactgcccctttcggccgcttgcaccgctctttccttcccttctttcgctttccagcgggcgctagggcgctggcaagtgtagcggtcacgctgcgcgtaaccaccacacccgccgcgcttaatgcgccgctacagggcgcgtcccattcgccatcgcccgcgatcccgcgaccgttcacatcgccagtgcgacgcgcattggtggtgtgggcggcgcgaattacgcggcgatgtcccgcgcagggtaagcggt

SEQ ID NO: 36 - DNA, 327 bp, Artificial sequence
  Artificial sequence descriptionβ synthetic DNA encoding thioredoxin in the plasmid pET32a+
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcc

SEQ ID NO: 37 - protein, 109 aa, Artificial sequence
  Artificial sequence descriptionβ thioredoxin protein encoded by the plasmid pET32a+
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 38 - DNA, 27 bp, Artificial sequence
  Artificial sequence descriptionβ upstream oligonucleotide amont for amplification of the DNAs encoding TME1 and TME2 from the constructs produced in pGEXKT, and insertion into pET32a+
gtgatatctgatctgtctggtggtggt

SEQ ID NO: 39 - DNA, 23 bp, Artificial sequence
  Artificial sequence descriptionβ downstream oligonucleotide for amplification of the DNA encoding TME1 from the construct produced in pGEXKT, and insertion into pET32a+
gaattcctaagcttcagcctgag

SEQ ID NO: 40 - DNA, 30 bp, Artificial sequence
  Artificial sequence descriptionβ downstream oligonucleotide for amplification of the DNAs encoding wild-type and mutant TME2 from the constructs produced in pGEXKT, and insertion into pET32a
gaattcttaagcttcagcctgagagatcag

SEQ ID NO: 41 - DNA, 5918 bp, Artificial sequence
  Artificial sequence description pET32a-DP-TME1 (complementary strand)
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcctaagcgtcaacaccagcgaacagcagcagaacaaccagaactttagcccagttaccaaccatagagaagtaagcgataccagccagaacaccccagtgagcaccagcgatcgggtcggatccacgcggaaccagaccaccaccaccaccagacagatccgattttggagatccagaaccagaaccggccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 42 - DNA, 513 bp, Artificial sequence
  Artificial sequence descriptionβ synthetic DNA encoding the TrX-DP-TME1 fusion protein in the vector pET32a-DP-TME1
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctccaaaatcggatctgtctggtggtggtggtggtctggttccgcgtggatccgacccgatcgctggtgctcactggggtgttctggctggtatcgcttacttctctatggttggtaactgggctaaagttctggttgttctgctgctgttcgctggtgttgacgcttag

SEQ ID NO: 43 - protein, 170 aa, Artificial sequence
  Artificial sequence descriptionβ TrX-DP-TME1 fusion protein encoded by the vector pET32a-DP-TME1
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGSPKSDLSGGGGGLVPRGSDPIAGAHWGVLAGIAYFSMVGNWAKVLVVLLLFAGVDA

SEQ ID NO: 44 - DNA, 5918 bp, Artificial sequence
  Artificial sequence descriptionβ vector pET32a-DP-TME1_G354L (complementary strand)
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcctaagcgtcaacaccagcgaacagcagcagaacaaccagaactttagcccagttaccaaccatagagaagtaagcgataccagccagaaccagccagtgagcaccagcgatcgggtcggatccacgcggaaccagaccaccaccaccaccagacagatccgattttggagatccagaaccagaaccggccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 45 - DNA, 513 bp, Artificial sequence
  Artificial sequence sythetic DNA encoding the TrX-DP-TME1_G354L fusion protein in the vector pET32a-DP-TME1_G354L
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctccaaaatcggatctgtctggtggtggtggtggtctggttccgcgtggatccgacccgatcgctggtgctcactggctggttctggctggtatcgcttacttctctatggttggtaactgggctaaagttctggttgttctgctgctgttcgctggtgttgacgcttag

SEQ ID NO: 46 - protein, 170 aa, Artificial sequence
  Artificial sequence descriptionβ TrX-DP-TME1_G354L fusion protein encoded by the vector pET32a-DP-TME1_G354L
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGSPKSDLSGGGGGLVPRGSDPIAGAHWLVLAGIAYFSMVGNWAKVLVVLLLFAGVDA

SEQ ID NO: 47 - DNA, 5918 bp, Artificial sequence
  Artificial sequence descriptionβ vector pET32a-DP-TME1_358L (complementary strand)
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcctaagcgtcaacaccagcgaacagcagcagaacaaccagaactttagcccagttaccaaccatagagaagtaagcgatcagagccagaacaccccagtgagcaccagcgatcgggtcggatccacgcggaaccagaccaccaccaccaccagacagatccgattttggagatccagaaccagaaccggccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 48 - DNA, 513 bp, Artificial sequence
  Artificial sequence descriptionβ synthetic DNA encoding the TrX-DP-TME1_G358L fusion protein in the vector pET32a-DP-TME1_G358L
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctccaaaatcggatctgtctggtggtggtggtggtctggttccgcgtggatccgacccgatcgctggtgctcactggggtgttctggctctgatcgcttacttctctatggttggtaactgggctaaagttctggttgttctgctgctgttcgctggtgttgacgcttag

SEQ ID NO: 49 - protein, 170 aa, Artificial sequence
  Artificial sequence descriptionβ TrX-DP-TME1_G358L fusion protein encoded by the vector pET32a-DP-TME1_G358L
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGSPKSDLSGGGGGLVPRGSDPIAGAHWGVLALIAYFSMVGNWAKVLVVLLLFAGVDA

SEQ ID NO: 50 - DNA, 5918 bp, Artificial sequence
  Artificial sequence descriptionβ vector pET32a-DP-TME1_G354&G358L (complementary strand)
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcctaagcgtcaacaccagcgaacagcagcagaacaaccagaactttagcccagttaccaaccatagagaagtaagcgatcagagccagaaccagccagtgagcaccagcgatcgggtcggatccacgcggaaccagaccaccaccaccaccagacagatccgattttggagatccagaaccagaaccggccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 51 - DNA, 513 bp, Artificial sequence
  Articial sequence descriptionβ synthetic DNA encoding the TrX-DP-TME1_G354&G358L fusion protein in the vector pET32a-DP-TME1_G354&G358L
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctccaaaatcggatctgtctggtggtggtggtggtctggttccgcgtggatccgacccgatcgctggtgctcactggctggttctggctctgatcgcttacttctctatggttggtaactgggctaaagttctggttgttctgctgctgttcgctggtgttgacgcttag

SEQ ID NO: 52 - protein, 170 aa, Artificial sequence
  Artificial sequence descriptionβ TrX-DP-TME1_G354&G358L fusion protein encoded by the vector pET32a-DP-TME_G354&G358L
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGSPKSDLSGGGGGLVPRGSDPIAGAHWLVLALIAYFSMVGNWAKVLVVLLLFAGVDA

SEQ ID NO: 53 - DNA, 5891 bp, Artificial sequence
  Artificial sequence descriptionβ vector pET32a-DP-TME2 (complementary strand)
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcctaagcttcagcctgagagatcagcagcatcatccacaggcaagagcaaacacgagcgtcagccagcagcaggaacagcagaacaacgtattccgggtcggatccacgcggaaccagaccaccaccaccaccagacagatcagatccagaaccagaaccggccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 54 - DNA, 486 bp, Artificial sequence
  Artificial sequence descriptionβ sythetic DNA encoding the fusion protein TrX-DP-TME2 in the vector pET32a-DP-TME2
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctgatctgtctggtggtggtggtggtctggttccgcgtggatccgacccggaatacgttgttctgctgttcctgctgctggctgacgctcgtgtttgctcttgcctgtggatgatgctgctgatctctcaggctgaagcttag

SEQ ID NO: 55 - protein, 161 aa, Artificial sequence
  Artificial sequence descriptionβ TrX-DP-TME2 fusion protein encoded by the vector pET32a-DP-TME2
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGSDLSGGGGGLVPRGSDPEYVVLLFLLLADARVCSCLWMMLLISQAEA

SEQ ID NO: 56 - DNA, 5891 bp, Artificial sequence
  Artificial sequence descriptionβ vector pET32a-DP-TME2_C731&733A (complementary strand)
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcctaagcttcagcctgagagatcagcagcatcatccacagagcagaagcaacacgagcgtcagccagcagcaggaacagcagaacaacgtattccgggtcggatccacgcggaaccagaccaccaccaccaccagacagatcagatccagaaccagaaccggccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 57 - DNA, 486 bp, Artificial sequence
  Artificial sequence descriptionβ DNA encoding the TrX-DP-TME2_C731&733A fusion protein in the vector pET32a-DP-TME2_C731&733A
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctgatctgtctggtggtggtggtggtctggttccgcgtggatccgacccggaatacgttgttctgctgttcctgctgctggctgacgctcgtgttgcttctgctctgtggatgatgctgctgatctctcaggctgaagcttag

SEQ ID NO: 58 - protein, 161 aa, Artificial sequence
  Artificial sequence descriptionβ TrX-DP-TME2_C731&733A fusion protein encoded by the vector pET32a-DP-TME2_C731&733A
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGSDLSGGGGGLVPRGSDPEYVVLLFLLLADARVASALWMMLLISQAEA

SEQ ID NO: 59 - DNA, 30 bp, Artificial sequence
  Artificial sequence description oligonucleotide PET998-AlwNI (+) for amplifying the  TrX-DP-TME1 cassette
ttcagtggctgtgcatgcaaggagatggcg

SEQ ID NO: 60 - DNA, 30 bp, Artificial sequence
  Artificial sequence description oligonucleotide PET998-AlwNI (-) for amplifying the TrX-DP-TME1 cassette
ttcagccactgctaagcgtcaacaccagcg

SEQ ID NO: 61 - DNA, 5914 bp, Artificial sequence
  Artificial sequence descriptionβ plasmid pGEXKT-DP-TME2+TrX-DP-TME1
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgtttgctcttgcctgtggatgatgctgctgatctctcaggctgaagcttaggaattcgcccttttcagtggctgtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctgatctgtctggtggtggtggtggtctggttccgcgtggatccgacccgatcgctggtgctcactggggtgttctggctggtatcgcttacttctctatggttggtaactgggctaaagttctggttgttctgctgctgttcgctggtgttgacgcttagcagtggctgaaaagggcgaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 62 - DNA, 5914 bp, Artifical sequence
  Artificial sequence descriptionβ plasmid pGEXKT-DP-TME2_C731&733A+TrX-DP-TME1
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgttgcttctgctctgtggatgatgctgctgatctctcaggctgaagcttaggaattcgcccttttcagccactgctaagcgtcaacaccagcgaacagcagcagaacaaccagaactttagcccagttaccaaccatagagaagtaagcgataccagccagaacaccccagtgagcaccagcgatcgggtcggatccacgcggaaccagaccaccaccaccaccagacagatcagatccagaaccagaaccggccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcacagccactgaaaagggcgaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 63 - protein, 15 aa, Artificial sequence
  Artificial sequence descriptionβ integrated sequence of pGEXKT
SDLSGGGGGLVPRGS

SEQ ID NO: 64 - DNA, 5082 bp, Artificial sequence
  Artificial sequence descriptionβ vector pGEXKT-DP-TME1_G354L
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccgatcgctggtgctcactggctggttctggctggtatcgcttacttctctatggttggtaactgggctaaagttctggttgttctgctgctgttcgctggtgttgacgcttaggaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 65 - protein, 271 aa, Artificial sequence
  Artificial sequence descriptionβ chimeric protein GSTkt-DP-TME1_G354L
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLSGGGGGLVPRGSDPIAGAHWLVLAGIAYFSMVGNWAKVLVVLLLFAGVDA

SEQ ID NO: 66 - DNA, 5082 bp, Artificial sequence
  Artificial sequence description vector pGEXKT-DP-TME1_G358L
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccgatcgctggtgctcactggggtgttctggctctgatcgcttacttctctatggttggtaactgggctaaagttctggttgttctgctgctgttcgctggtgttgacgcttaggaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 67 - protein, 271 aa, Artificial sequence
  Artificial sequence descriptionβ chimeric protein pGEXkt-DP-TME1_G358L
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLSGGGGGLVPRGSDPIAGAHWGVLALIAYFSMVGNWAKVLVVLLLFAGVDA

SEQ ID NO: 68 - DNA, 5082 bp, Artificial sequence
  Artificial sequence descriptionβ vector pGEXKT-DP-TME1_G354&358L
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccgatcgctggtgctcactggctggttctggctctgatcgcttacttctctatggttggtaactgggctaaagttctggttgttctgctgctgttcgctggtgttgacgcttaggaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 69 - protein, 271 aa, Artificial sequence
  Artificial sequence description pGEXkt-DP-TME1_G354&358L
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLSGGGGGLVPRGSDPIAGAHWLVLALIAYFSMVGNWAKVLVVLLLFAGVDA

SEQ ID NO: 70 - DNA, 5914 bp, Artificial sequence
  Artificial sequence descriptionβ vector pGEXKT-DP-TME2 + TrX-DP-TME1_G354L
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgtttgctcttgcctgtggatgatgctgctgatctctcaggctgaagcttaggaattcgcccttttcagtggctgtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctgatctgtctggtggtggtggtggtctggttccgcgtggatccgacccgatcgctggtgctcactggctggttctggctggtatcgcttacttctctatggttggtaactgggctaaagttctggttgttctgctgctgttcgctggtgttgacgcttagcagtggctgaaaagggcgaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 71 - DNA, 5914 bp, Artificial sequence
  Artificial sequence descriptionβ vector pGEXKT-DP-TME2 + TrX-DP-TME1_G358L
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgtttgctcttgcctgtggatgatgctgctgatctctcaggctgaagcttaggaattcgcccttttcagtggctgtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctgatctgtctggtggtggtggtggtctggttccgcgtggatccgacccgatcgctggtgctcactggggtgttctggctctgatcgcttacttctctatggttggtaactgggctaaagttctggttgttctgctgctgttcgctggtgttgacgcttagcagtggctgaaaagggcgaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 72 - DNA, 5914 bp, Artificial sequence
  Artificial sequence descriptionβ vector pGEXKT-DP-TME2 + TrX-DP-TME1_G354&358L
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgtttgctcttgcctgtggatgatgctgctgatctctcaggctgaagcttaggaattcgcccttttcagtggctgtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctgatctgtctggtggtggtggtggtctggttccgcgtggatccgacccgatcgctggtgctcactggctggttctggctctgatcgcttacttctctatggttggtaactgggctaaagttctggttgttctgctgctgttcgctggtgttgacgcttagcagtggctgaaaagggcgaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 73 - DNA, 5914 bp, Artificial sequence
  Artificial sequence descriptionβ vector pGEXKT-DP-TME2_C731&C733A + TrX-DP-TME1_G354L
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgttgcttctgctctgtggatgatgctgctgatctctcaggctgaagcttaggaattcgcccttttcagtggctgtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctgatctgtctggtggtggtggtggtctggttccgcgtggatccgacccgatcgctggtgctcactggctggttctggctggtatcgcttacttctctatggttggtaactgggctaaagttctggttgttctgctgctgttcgctggtgttgacgcttagcagtggctgaaaagggcgaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 74 - DNA, 5914 bp, Artificial sequence
  Artificial sequence descriptionβ vector pGEXKT-DP-TME2_C731&C733A + TrX-DP-TME1_G358L
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgttgcttctgctctgtggatgatgctgctgatctctcaggctgaagcttaggaattcgcccttttcagtggctgtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctgatctgtctggtggtggtggtggtctggttccgcgtggatccgacccgatcgctggtgctcactggggtgttctggctctgatcgcttacttctctatggttggtaactgggctaaagttctggttgttctgctgctgttcgctggtgttgacgcttagcagtggctgaaaagggcgaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 75 - DNA, 5914 bp, Artificial sequence
  Artificial sequence descriptionβ vector pGEXKT-DP-TME2_C731&C733A + TrX-DP-TME1_G354&358L
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctgtctggtggtggtggtggtctggttccgcgtggatccgacccggaatacgttgttctgctgttcctgctgctggctgacgctcgtgttgcttctgctctgtggatgatgctgctgatctctcaggctgaagcttaggaattcgcccttttcagtggctgtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggatctgatctgtctggtggtggtggtggtctggttccgcgtggatccgacccgatcgctggtgctcactggctggttctggctctgatcgcttacttctctatggttggtaactgggctaaagttctggttgttctgctgctgttcgctggtgttgacgcttagcagtggctgaaaagggcgaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 76 - DNA, 74 bp, Artificial sequence
  Artificial sequence description oligonucleotide OL11(+) for generating the DNA encoding TME1
atgccatatgatcgctggtgctcactggggtgttctggctggtatcgcttacttctctatggttggtaactggg

SEQ ID NO: 77 - DNA, 79 bp, Artificial sequence
  Artificial sequence description oligonucleotide OL12(-) for generating the DNA encoding TME1
gcatatcgatctaagcgtcaacaccagcgaacagcagcagaacaaccagaactttagcccagttaccaaccatagagaa

SEQ ID NO: 78 - DNA, 28 bp, Artificial sequence
  Artificial sequence description oligonucleotide OL17(+) for amplifying the DNA encoding TME1 for insertion into the plasmid pGEXKT
ggatccgacccgatggaatacgttgttc

SEQ ID NO: 79 - DNA, 68 bp, Artificial sequence
  Artificial sequence description oligonucleotide OL21(+) for generating the DNA encoding TME2
catatggaatacgttgttctgctgttcctgctgctggctgacgctcgtgtttgctcttgcctgtggat

SEQ ID NO: 80 - DNA, 57 bp, Artificial sequence
  Artificial sequence description oligonucleotide OL22(-) for generating the DNA encoding TME2
aagcttaagcttcagcctgagagatcagcagcatcatccacaggcaagagcaaacac

SEQ ID NO: 81 - DNA, 25 bp, Artificial sequence
  Artificial sequence description oligonucleotide OL27(+) for amplifying the DNA encoding TME2 for insertion into the plasmid pGEXKT
ggatccgacccggaatacgttgttc